SEQUENCE LISTING (23 sequences)

SEQ ID NO: 1 - DNA, 369 bp, Unknown
  Description of Unknown Cyanobacteria
  gene (1)..(369): Wild Type Cyanoglobin gene
tcggtttaaaacatcatttcgatgggatagagccccgacaatcgccccaacttcgtcaattaaattttgttcgatacctaactctttgagagtattgatcaaattttctgcgatcgcatcaaaatgaacatccgttaaacccatattatctactaaatgtttatgggattctcgcatatctctaccagaaaaagtatcaggacctccaaaagcataagtcataaaagccttttgatgagctttttgtttcttcatatccgtattggcaaagaaataattaactcggtcatcctttaacaccttatcatagaacttatcgactgcaatatctacagcttctttaccgcctaatttttcgtacaaactact

SEQ ID NO: 2 - protein, 124 aa, Unknown
  Description of Unknown Cyanobacteria
  NP_BIND (1)..(124): Wild type Cyanoglobin protein
MSSLYEKLGGKEAVDIAVDKFYDKVLKDDRVNYFFANTDMKKQKAHQKAFMTYAFGGPDTFSGRDMRESHKHLVDNMGLTDVHFDAIAENLINTLKELGIEQNLIDEVGAIVGALSHRNDVLNR

SEQ ID NO: 3 - DNA, 1227 bp, Escherichia coli
  gene (1)..(1227): Wild type HemA gene
atggattataacttggcattagatactgcattgaaccgtttacacactgaagggcgttatcgcacattcatcgacatcgaacgccgcaagggcgctttcccgaaggctatgtggcgtaagccggatggatctgagaaagagattacggtctggtgcggcaacgattacttggggatgggacagcacccggtagttcttggagcaatgcacgaggcacttgactcgacaggtgcggggtctgggggtacccgcaatatctccggcacaacattatatcataaacgtctggaagcagaactggcagatttacatgggaaggaagcagcgttagtctttagctcagcatacatcgcaaacgacgctacgttgtctacgttgcctcaacttattcccggacttgtcattgtctccgacaagctgaatcatgctagtatgatcgaagggatccgccgctcgggaactgaaaaacacatcttcaaacataatgacctggatgatttgcgccgcattcttaccagcattgggaaggatcgcccaattttggttgcgttcgagagtgtatatagtatggacggtgacttcggacgcattgaggaaatttgcgacatcgcagatgaatttggagcattaaagtacattgatgaagtacacgcggtgggaatgtatggaccgcgcggtggtggtgttgctgaacgtgacggactgatggaccgcatcgacattattaacggcacgctgggtaaagcctacggagttttcggagggtatattgccgcctcttctaagatgtgtgatgcggtacgctcatatgctccgggatttattttctccacttctctgccgccggtggtcgccgctggggccgctgcttccgttcgccacctgaagggtgacgtagaattacgtgagaagcaccagacacaggcgcgtattttaaagatgcgtttgaagggattgggtctgcctattattgatcacgggtctcatatcgtccctgtacacgtcggcgatccggtccactgcaagatgatttctgacatgctgctggaacacttcggcatttatgtacagcctatcaacttcccgacagttcctcgtggaaccgagcgcttgcgtttcactccttcacctgttcacgactctgggatgatcgatcacttagttaaagcaatggacgtcttatggcagcactgtgctcttaatcgtgccgaggtagtggctctcgag

SEQ ID NO: 4 - protein, 409 aa, Escherichia coli
  PEPTIDE (2)..(409): Wild type HemA protein
MDYNLALDTALNRLHTEGRYRTFIDIERRKGAFPKAMWRKPDGSEKEITVWCGNDYLGMGQHPVVLGAMHEALDSTGAGSGGTRNISGTTLYHKRLEAELADLHGKEAALVFSSAYIANDATLSTLPQLIPGLVIVSDKLNHASMIEGIRRSGTEKHIFKHNDLDDLRRILTSIGKDRPILVAFESVYSMDGDFGRIEEICDIADEFGALKYIDEVHAVGMYGPRGGGVAERDGLMDRIDIINGTLGKAYGVFGGYIAASSKMCDAVRSYAPGFIFSTSLPPVVAAGAAASVRHLKGDVELREKHQTQARILKMRLKGLGLPIIDHGSHIVPVHVGDPVHCKMISDMLLEHFGIYVQPINFPTVPRGTERLRFTPSPVHDSGMIDHLVKAMDVLWQHCALNRAEVVALE

SEQ ID NO: 5 - DNA, 1224 bp, Artificial Sequence
  Synthetic
  gene (1)..(1224): Synthetic HemA (Codon optimized for E. coli)
atggattataacttggcattagatactgcattgaaccgtttacacactgaagggcgttatcgcacattcatcgacatcgaacgccgcaagggcgctttcccgaaggctatgtggcgtaagccggatggatctgagaaagagattacggtctggtgcggcaacgattacttggggatgggacagcacccggtagttcttggagcaatgcacgaggcacttgactcgacaggtgcggggtctgggggtacccgcaatatctccggcacaacattatatcataaacgtctggaagcagaactggcagatttacatgggaaggaagcagcgttagtctttagctcagcatacatcgcaaacgacgctacgttgtctacgttgcctcaacttattcccggacttgtcattgtctccgacaagctgaatcatgctagtatgatcgaagggatccgccgctcgggaactgaaaaacacatcttcaaacataatgacctggatgatttgcgccgcattcttaccagcattgggaaggatcgcccaattttggttgcgttcgagagtgtatatagtatggacggtgacttcggacgcattgaggaaatttgcgacatcgcagatgaatttggagcattaaagtacattgatgaagtacacgcggtgggaatgtatggaccgcgcggtggtggtgttgctgaacgtgacggactgatggaccgcatcgacattattaacggcacgctgggtaaagcctacggagttttcggagggtatattgccgcctcttctaagatgtgtgatgcggtacgctcatatgctccgggatttattttctccacttctctgccgccggtggtcgccgctggggccgctgcttccgttcgccacctgaagggtgacgtagaattacgtgagaagcaccagacacaggcgcgtattttaaagatgcgtttgaagggattgggtctgcctattattgatcacgggtctcatatcgtccctgtacacgtcggcgatccggtccactgcaagatgatttctgacatgctgctggaacacttcggcatttatgtacagcctatcaacttcccgacagttcctcgtggaaccgagcgcttgcgtttcactccttcacctgttcacgactctgggatgatcgatcacttagttaaagcaatggacgtcttatggcagcactgtgctcttaatcgtgccgaggtagtggcttga

SEQ ID NO: 6 - protein, 407 aa, Artificial Sequence
  Synthetic
  PEPTIDE (1)..(407): Synthetic HemA protein (Codon optimized for E. coli)
MDYNLALDTALNRLHTEGRYRTFIDIERRKGAFPKAMWRKPDGSEKEITVWCGNDYLGMGQHPVVLGAMHEALDSTGAGSGGTRNISGTTLYHKRLEAELADLHGKEAALVFSSAYIANDATLSTLPQLIPGLVIVSDKLNHASMIEGIRRSGTEKHIFKHNDLDDLRRILTSIGKDRPILVAFESVYSMDGDFGRIEEICDIADEFGALKYIDEVHAVGMYGPRGGGVAERDGLMDRIDIINGTLGKAYGVFGGYIAASSKMCDAVRSYAPGFIFSTSLPPVVAAGAAASVRHLKGDVELREKHQTQARILKMRLKGLGLPIIDHGSHIVPVHVGDPVHCKMISDMLLEHFGIYVQPINFPTVPRGTERLRFTPSPVHDSGMIDHLVKAMDVLWQHCALNRAEVVA

SEQ ID NO: 7 - DNA, 1224 bp, Artificial Sequence
  Synthetic
  gene (1)..(1224): Synthetic HemA (Codon optimized for Cyanobacteria)
atggattataatttagctttagatactgctttaaatagattacatactgaaggtagatatagaacttttattgatattgaaagaagaaaaggtgcttttcctaaagctatgtggagaaaacctgatggttctgaaaaagaaattactgtttggtgtggtaatgattatttaggtatgggtcaacatcctgttgttttaggtgctatgcatgaagctttagattctactggtgctggttctggtggtactagaaatatttctggtactactttatatcataaaagattagaagctgaattagctgatttacatggtaaagaagctgctttagttttttcttctgcttatattgctaatgatgctactttatctactttacctcaattaattcctggtttagttattgtttctgataaattaaatcatgcttctatgattgaaggtattagaagatctggtactgaaaaacatatttttaaacataatgatttagatgatttaagaagaattttaacttctattggtaaagatagacctattttagttgcttttgaatctgtttattctatggatggtgattttggtagaattgaagaaatttgtgatattgctgatgaatttggtgctttaaaatatattgatgaagttcatgctgttggtatgtatggtcctagaggtggtggtgttgctgaaagagatggtttaatggatagaattgatattattaatggtactttaggtaaagcttatggtgtttttggtggttatattgctgcttcttctaaaatgtgtgatgctgttagatcttatgctcctggttttattttttctacttctttacctcctgttgttgctgctggtgctgctgcttctgttagacatttaaaaggtgatgttgaattaagagaaaaacatcaaactcaagctagaattttaaaaatgagattaaaaggtttaggtttacctattattgatcatggttctcatattgttcctgttcatgttggtgatcctgttcattgtaaaatgatttctgatatgttattagaacattttggtatttatgttcaacctattaattttcctactgttcctagaggtactgaaagattaagatttactccttctcctgttcatgattctggtatgattgatcatttagttaaagctatggatgttttatggcaacattgtgctttaaatagagctgaagttgttgcttga

SEQ ID NO: 8 - protein, 407 aa, Artificial Sequence; identical to SEQ ID NO: 6
  Synthetic
  PEPTIDE (1)..(407): Synthetic HemA protein (Codon optimized for Cyanobacteria)

SEQ ID NO: 9 - DNA, 1836 bp, Artificial Sequence
  Synthetic
  gene (1)..(1836): FeoB (Codon optimized for Cyanobacteria)
atgtcttgtcattctgctaccacgaaagttttaccaaattctcaaaagcgtattgccgtaattggacaaccgaatactggaaaatcaaccttttttaatcgtttaactgggagtaatgcttctgtaggcaattggtctggtataactgtggatttactacaggcagaaattaatctgaataatgaagtggtggaagtggtagatttacccggtatttatgattttaatggcttttctgatgatgaaaaggtggtgcagaggtttttggaaacttttcccctcgatttagctattattattatcaacgctactcaaattgatcgcaccttaagattaatattagagatgaaaaggttaggattaccttgcgtcgtaatgttaaatatggcagatgaagccaaacgctatggagtagaaataaatcactatcaactgcaagagattttagggataccttgtttttcaattagtgccaaatatggtaaaggtttttcccaagctgttaggggcatagaaaaggcactatccacccaagaggattctttcattcttaaagatgttcatgattttctctccacgaatcctgtaacagagacagaaattaactcgattttggagaaaaccgtcaaagtaccccctgaagaaattaaaacgattacggataaactcgatcgcattttacttcatcctattttcggaataccacttttttttatttccatgttaggaatattctttctaatttggtatctcggtttaccctctcaagacattatgggaaatatcaccgattggattagtgggcaaattatcgagccaactattcaaccactgcccgaaatattgcaaaattttctcctcaatggagtttggttaggtgtagcaactgtagcttcttttgtacctttaatcgttctctttttctttgtaatggcggcggtagaagatagtggttatctagctcgatcagcttacctcatggatgctttaatggaaaaattagggctagatggtagggcttttgtcatgcaaatgatgggatttggctgtaatgttcctgctttaatgggtacaagagtaattcgatcgcaacctatgcgttggttatcaatgttaattataccttttgccctttgttctgcaagactagcagtatttgtttttattattgccgctattttccctcccaatttaggaccgattattcttttttctctctatttactaagttttattgctagtttttttgttgcttttgtatttagtaaaactaaagaatttcaaagtcaagaaccttttattattgaacttcctccctatagattacccactttaagacaagtactattaaggggatggggggaagtaaaagaatttttgcgaagggcaactaactttattatcatcgggtgtgtagcagtatggtttttaacttattttcctgaaaacagcataggattagagacatggggaggacaactaggaattttattacaacctattatgcaaccgattggcattgatccctatttaactttatctttaatttttggctttattgctaaagaaattgttgtgggttcatttgccacgatttatggtttaagttcatcggcattaactcaacatattgccctaactataactcctgcggcggcagtttctttttgtatcttttgcttattatatactccttgtttatccaccgttgccaccattaaagcagagtccaaatcgacaatttttactttattttctttgcttttttctctagtttatgcttggatatgtgcttttattttctatcatctggcactaacattatttcctatttaa

SEQ ID NO: 10 - protein, 611 aa, Artificial Sequence
  Synthetic
  PEPTIDE (1)..(611): FeoB protein (Codon optimized for Cyanobacteria)
MSCHSATTKVLPNSQKRIAVIGQPNTGKSTFFNRLTGSNASVGNWSGITVDLLQAEINLNNEVVEVVDLPGIYDFNGFSDDEKVVQRFLETFPLDLAIIIINATQIDRTLRLILEMKRLGLPCVVMLNMADEAKRYGVEINHYQLQEILGIPCFSISAKYGKGFSQAVRGIEKALSTQEDSFILKDVHDFLSTNPVTETEINSILEKTVKVPPEEIKTITDKLDRILLHPIFGIPLFFISMLGIFFLIWYLGLPSQDIMGNITDWISGQIIEPTIQPLPEILQNFLLNGVWLGVATVASFVPLIVLFFFVMAAVEDSGYLARSAYLMDALMEKLGLDGRAFVMQMMGFGCNVPALMGTRVIRSQPMRWLSMLIIPFALCSARLAVFVFIIAAIFPPNLGPIILFSLYLLSFIASFFVAFVFSKTKEFQSQEPFIIELPPYRLPTLRQVLLRGWGEVKEFLRRATNFIIIGCVAVWFLTYFPENSIGLETWGGQLGILLQPIMQPIGIDPYLTLSLIFGFIAKEIVVGSFATIYGLSSSALTQHIALTITPAAAVSFCIFCLLYTPCLSTVATIKAESKSTIFTLFSLLFSLVYAWICAFIFYHLALTLFPI

SEQ ID NO: 11 - DNA, 1308 bp, Artificial Sequence
  Synthetic
  gene (1)..(1308): HemL (Codon optimized for Cyanobacteria)
ttggtaaacacagtatcgttcaaaactactaaatccgatgaaatctttgccgccgcccaaaaattaatgccgggaggagttaattctcctgtaagagcctttaaatctgtcggtggacaacctattgtttttgaccgtgtaaaaggtgcatatatttgggatgtggacgaaaataaatatattgactatgttgggacttggggacctgcaatttgtggtcatgctcatcctgaagtcatcaaatctttaagtgaggctttggaaaaaggtacaagttttggcgctccttgttatttagaaaatatcctcgccgaaatggtgattgatgcagtaccaagcattgagatggtgcgttttgttaactctggtacagaagcctgtatttctgttttaagattgatgagagcctatacaggtagaaacaaaatcatcaagtttgaaggttgttatcatggccatggcgatatgtttttagttaaggcaggttctggagttgctaccctcggcttaccagattctcccggagtacctaaaagcgttacatcagatactttgactgcaccttataacgatttagaagcggtgaaaaaattatttgccgataatcctgatgaaattgcgggggttattcttgagcctgtagtgggtaactctggttttattgtccctgatgcgggttttttagagggtttaagattaattactgagcagaatggtgctttattggtattcgatgaagtaatgactggtttccgcatatcttatggtggcgctcaagctaaattcggtattactcccgatttaacaactttaggaaaagttattggcggtggtttacctgtgggtgcttatggtggtaaaaaggaaatcatggcaatggttgctccttctggccccatgtatcaggctggtactttgtctggtaatcctttagctatgacagcaggaattaaaacccttgaattattacaaaaacccggcacttatgaatatttggaagaaattacccataaactaataagtggtttattaactattgctaaaaatactggtcataaggttactggcggtaatattagtggtatgtttggcatgtttttcactggtgaagaagttcataattatgatgatgcgaaaaaagctgacaccagtaaattcgcccgttttcatagaggaatgcttgagagaggagtatatttagcaccctcccagtttgaggctggatttacttctattgctcacaccgaagaggatataaaccaaactttagctatggcggaagaagttttgaaagaaattcctgcctaa

SEQ ID NO: 12 - protein, 435 aa, Artificial Sequence
  Synthetic
  PEPTIDE (1)..(435): HemL protein (Codon optimized for Cyanobacteria)
MVNTVSFKTTKSDEIFAAAQKLMPGGVNSPVRAFKSVGGQPIVFDRVKGAYIWDVDENKYIDYVGTWGPAICGHAHPEVIKSLSEALEKGTSFGAPCYLENILAEMVIDAVPSIEMVRFVNSGTEACISVLRLMRAYTGRNKIIKFEGCYHGHGDMFLVKAGSGVATLGLPDSPGVPKSVTSDTLTAPYNDLEAVKKLFADNPDEIAGVILEPVVGNSGFIVPDAGFLEGLRLITEQNGALLVFDEVMTGFRISYGGAQAKFGITPDLTTLGKVIGGGLPVGAYGGKKEIMAMVAPSGPMYQAGTLSGNPLAMTAGIKTLELLQKPGTYEYLEEITHKLISGLLTIAKNTGHKVTGGNISGMFGMFFTGEEVHNYDDAKKADTSKFARFHRGMLERGVYLAPSQFEAGFTSIAHTEEDINQTLAMAEEVLKEIPA

SEQ ID NO: 13 - DNA, 1158 bp, Artificial Sequence
  Synthetic
  gene (1)..(1158): HemH (Codon optimized for Cyanobacteria)
gtggtagttactccggaaaaaactattgaatcaaattcagtctcacctgctacagatagagtagccgttattcttatgggatatggagaggtggaaagttatgatgattttgccaactataatgaacaagccttaaacctacttacggcaaaatttgcccctgtgccaacatggttatatcccccccttgctaaattgttagcggttttcgatttacatgaatggagtcatcaacacggcaattttgtttctcctcataatgctattttcgagcgtcaaagagcaaaaattgagcatcagttgcaacacacttggggagaaaaagtcaaagtatttaaggctttcaacttctgtaaacctcatttaccctatcaagttttagagcaggttaaagctgaaggatttgagaaaattctcatttatcccctcttagtagttgattctatcttcaccagtggaattgcgatcgagcaaataaacgaggcgttagcacaaaaagacaaaaaagaagaccattgggttaaagccttacgttatataccttctttctacaataaaccagagtatatcgatttattagctaaattagtagaagaagaaatcgacgcaaaattaagcaatgcttatttaccttcagaaattggtattgttttaatgaatcacggttgccctcatgaagcaaaaggatttacttcgggtattgatgaaagtcaagccttatatgaattggtaagagaaaaactcatttatcgttatcccttaatttccgtaggttggctcaaccatcaaacccctctaattaaatggactcaacctaatgcggagttagcaggaagaaacttgattgagttaggggcaaaagccttaatatttatgcccattggctttgcaacggaaaaccatgaaaccttgttagatgtagaacacattattcaagcattatacgttaaatatcccgatgtcaattatgtccagatggaatgcgttaacgataatgaagaattttgtgctatggcggcacaatgggttaatcctttgattgaatccttattaacggaagaaggggctgtaattaatcctagttttgccaaaacccattatcatggtcatcatcaccacgatcatgatcataatcacaatcatcatcaccatcattaa

SEQ ID NO: 14 - protein, 385 aa, Artificial Sequence
  Synthetic
  PEPTIDE (1)..(385): HemH protein (Codon optimized for Cyanobacteria)
MVVTPEKTIESNSVSPATDRVAVILMGYGEVESYDDFANYNEQALNLLTAKFAPVPTWLYPPLAKLLAVFDLHEWSHQHGNFVSPHNAIFERQRAKIEHQLQHTWGEKVKVFKAFNFCKPHLPYQVLEQVKAEGFEKILIYPLLVVDSIFTSGIAIEQINEALAQKDKKEDHWVKALRYIPSFYNKPEYIDLLAKLVEEEIDAKLSNAYLPSEIGIVLMNHGCPHEAKGFTSGIDESQALYELVREKLIYRYPLISVGWLNHQTPLIKWTQPNAELAGRNLIELGAKALIFMPIGFATENHETLLDVEHIIQALYVKYPDVNYVQMECVNDNEEFCAMAAQWVNPLIESLLTEEGAVINPSFAKTHYHGHHHHDHDHNHNHHHHH

SEQ ID NO: 15 - protein, 426 aa, Artificial Sequence
  Synthetic
  PEPTIDE (1)..(426): HemL protein (Codon optimized for E. coli)
MSKSENLYSAARELIPGGVNSPVRAFTGVGGTPLFIEKADGAYLYDVDGKAYIDYVGSWGPMVLGHNHPAIRNAVIEAAERGLSFGAPTEMEVKMAQLVTELVPTMDMVRMVNSGTEATMSAIRLARGFTGRDKIIKFEGCYHGHADCLLVKAGSGALTLGQPNSPGVPADFAKYTLTCTYNDLASVRAAFEQYPQEIACIIVEPVAGNMNCVPPLPEFLPGLRALCDEFGALLIIDEVMTGFRVALAGAQDYYGVVPDLTCLGKIIGGGMPVGAFGGRRDVMDALAPTGPVYQAGTLSGNPIAMAAGFACLNEVAQPGVHETLDELTTRLAEGLLEAAEEAGIPLVVNHVGGMFGIFFTDAESVTCYQDVMACDVERFKRFFHMMLDEGVYLAPSAFEAGFMSVAHSMEDINNTIDAARRVFAKL

SEQ ID NO: 16 - DNA, 1281 bp, Artificial Sequence
  Synthetic
  gene (1)..(1281)
atgagtaagtctgaaaatctttacagcgcagcgcgcgagctgatccctggcggtgtgaactcccctgttcgcgcctttactggcgtgggcggcactccactgtttatcgaaaaagcggacggcgcttatctgtacgatgttgatggcaaagcctatatcgattatgtcggttcctgggggccgatggtgctgggccataaccatccggcaatccgcaatgccgtgattgaagccgccgagcgtggtttaagctttggtgcaccaaccgaaatggaagtgaaaatggcgcaactggtgaccgaactggtcccgaccatggatatggtgcgcatggtgaactccggcactgaagcgaccatgagcgccatccgcctggcccgtggttttaccggtcgcgacaaaattattaaatttgaagggtgttaccatggtcacgctgactgcctgctggtgaaagccggttctggcgcactcacgttaggccagccaaactcgccgggcgttccggcagatttcgccaaatataccttaacctgtacttataatgatctggcttctgtacgcgccgcatttgagcaatacccgcaagagattgcctgtattatcgtcgagccggtggcaggcaatatgaactgtgttccgccgctgccagagttcctgccaggtctgcgcgcgctgtgcgacgaatttggcgcgttgctgatcatcgatgaagtgatgaccggtttccgcgtagcgctagctggcgcacaggattattacggcgtagtgccagatttaacctgcctcggcaaaatcatcggcggtggaatgccggtaggcgcattcggtggtcgtcgtgatgtaatggatgcgctggccccgacgggtccggtctatcaggcgggtacgctttccggtaacccgattgcgatggcagcgggtttcgcctgtctgaatgaagtcgcgcagccgggcgttcacgaaacgctggatgagctgacaacacgtctggcagaaggtctgctggaagcggcagaagaagccggaattccgctggtcgttaaccacgttggcggcatgttcggtattttctttaccgacgccgagtccgtgacgtgctatcaggatgtgatggcctgtgacgtggaacgctttaagcgtttcttccatatgatgctggacgaaggtgtttacctggcaccgtcagcgtttgaagcgggctttatgtccgtggcgcacagcatggaagatatcaataacaccatcgatgctgcacgtcgggtgtttgcgaagttgtga

SEQ ID NO: 17 - protein, 320 aa, Artificial Sequence
  Synthetic
  PEPTIDE (1)..(320): HemH protein (Codon optimized for E. coli)
MRQTKTGILLANLGTPDAPTPEAVKRYLKQFLSDRRVVDTSRLLWWPLLRGVILPLRSPRVAKLYASVWMEGGSPLMVYSRQQQQALAQRLPEMPVALGMSYGSPSLESAVDELLAEHVDHIVVLPLYPQFSCSTVGAVWDELARILARKRSIPGISFIRDYADNHDYINALANSVRASFAKHGEPDLLLLSYHGIPQRYADEGDDYPQRCRTTTRELASALGMAPEKVMMTFQSRFGREPWLMPYTDETLKMLGEKGVGHIQVMCPGFAADCLETLEEIAEQNREVFLGAGGKKYEYIPALNATPEHIEMMANLVAAYR

SEQ ID NO: 18 - DNA, 963 bp, Artificial Sequence
  Synthetic
  gene (1)..(963): HemH (Codon optimized for E. coli)
atgcgtcagactaaaaccggtatcctgctggcaaacctgggtacgcccgatgcccccacacctgaagcggtaaaacgctatctgaaacaatttttaagcgacagacgcgtggttgatacctcacggttgttatggtggccattgctgcgcggcgtgattttgccgctgcgctcgccgcgtgtggcgaagctgtatgcctctgtctggatggaaggtggctcgccgctgatggtttacagccgccagcaacagcaggcgctggcacaacgtttaccggagatgcccgtagcgctgggaatgagctacggctcgccatcactggaaagcgccgtagatgaactcctggcagagcatgtagatcatattgtggtgctgccgctttatccgcaattctcctgttctacggtcggtgcggtatgggatgaactggcacgcattctggcgcgcaaacgtagcattccggggatatcgtttatacgtgattacgccgataaccacgattacattaatgcactggcgaacagcgtacgcgcttcttttgccaaacatggcgaaccggatctgctactgctctcttatcatggcattccccagcgttatgcagatgaaggcgatgattacccgcaacgttgccgcacaacgactcgtgaactggcttccgcattggggatggcaccggaaaaagtgatgatgacctttcagtcgcgctttggtcgggaaccctggctgatgccttataccgacgaaacgctgaaaatgctcggagaaaaaggcgtaggtcatattcaggtgatgtgcccgggctttgctgcggattgtctggagacgctggaagagattgccgagcaaaaccgtgaggtcttcctcggtgccggcgggaaaaaatatgaatatattccggcgcttaatgccacgccggaacatatcgaaatgatggctaatcttgttgccgcgtatcgctaa

SEQ ID NO: 19 - protein, 773 aa, Artificial Sequence
  Synthetic
  PEPTIDE (1)..(773): FeoB protein (Codon optimized for E. coli)
MKKLTIGLIGNPNSGKTTLFNQLTGSRQRVGNWAGVTVERKEGQFSTTDHQVTLVDLPGTYSLTTISSQTSLDEQIACHYILSGDADLLINVVDASNLERNLYLTLQLLELGIPCIVALNMLDIAEKQNIRIEIDALSARLGCPVIPLVSTRGRGIEALKLAIDRYKANENVELVHYAQPLLNEADSLAKVMPSDIPLKQRRWLGLQMLEGDIYSRAYAGEASQHLDAALARLRNEMDDPALHIADARYQCIAAICDVVSNTLTAEPSRFTTAVDKIVLNRFLGLPIFLFVMYLMFLLAINIGGALQPLFDVGSVALFVHGIQWIGYTLHFPDWLTIFLAQGLGGGINTVLPLVPQIGMMYLFLSFLEDSGYMARAAFVMDRLMQALGLPGKSFVPLIVGFGCNVPSVMGARTLDAPRERLMTIMMAPFMSCGARLAIFAVFAAAFFGQNGALAVFSLYMLGIVMAVLTGLMLKYTIMRGEATPFVMELPVYHVPHVKSLIIQTWQRLKGFVLRAGKVIIIVSIFLSAFNSFSLSGKIVDNINDSALASVSRVITPVFKPIGVHEDNWQATVGLFTGAMAKEVVVGTLNTLYTAENIQDEEFNPAEFNLGEELFSAIDETWQSLKDTFSLSVLMNPIEASKGDGEMGTGAMGVMDQKFGSAAAAYSYLIFVLLYVPCISVMGAIARESSRGWMGFSILWGLNIAYSLATLFYQVASYSQHPTYSLVCILAVILFNIVVIGLLRRARSRVDIELLATRKSVSSCCAASTTGDCH

SEQ ID NO: 20 - DNA, 2322 bp, Artificial Sequence
  Synthetic
  gene (1)..(2322): FeoB gene (Codon optimized for E. coli)
atgaaaaaattaaccattggcttaattggtaatccaaattctggcaagacaacgttatttaaccagctcactggctcacgtcagcgtgtaggtaactgggctggcgttaccgtcgaacgtaaagaagggcaattctccaccaccgatcatcaggtcacgctggtggacctgcccggcacctattctctgaccaccatctcatcgcagacctcgctcgatgagcaaatcgcctgtcactacattttgagtggcgacgccgacctgctgattaacgtggtggatgcgtctaaccttgagcgtaacctgtacctgacgctacaactgctggaactcggcattccctgcattgtggcactgaacatgctcgacattgccgagaagcaaaatattcgtattgaaattgatgctctgtcggcgcgtctgggctgtccggtgatcccgctggtttcaacccgtggtcgcggtattgaagcgctcaagctggcgattgatcgctataaagctaacgagaatgtggaactggtgcattacgcacagccgctgctcaacgaagcagattcactggcaaaagtgatgccttccgacatcccgctgaaacaacgtcgctggctgggcctgcaaatgctggaaggcgatatctacagccgcgcctacgccggtgaagcgtcgcagcatctggatgccgccctcgcccgtctgcgtaatgagatggacgatccggcgctgcacattgccgatgcgcgttaccagtgcattgctgccatctgtgatgtggtaagcaacaccctgacggcagaacccagccgtttcaccactgcggtagataaaatcgtgctcaaccgtttcctcggtctgccgattttcctctttgtgatgtacctgatgttcctgctggctatcaacatcggcggggcgttacagccgctgtttgacgtcggctccgtggcgctatttgtgcatggtattcaatggattggctacacgctccacttcccggactggctgactatcttcctcgcccagggcctgggtggcggcattaacaccgtgctgccactggtgccgcagattggcatgatgtacctgttcctctccttccttgaggactccgggtatatggcgcgtgcggcgtttgtgatggaccgtctgatgcaggcgctgggcttgccggggaaatcctttgtgccgctgatcgtcggtttcggttgtaacgtaccgtcggtaatgggtgcacgtacgcttgatgcaccgcgtgaacgtctgatgaccatcatgatggcaccgtttatgtcctgcggcgcgcgtctggctatcttcgcagtattcgcggctgccttcttcgggcagaacggtgcgctggcggtcttctcgctgtatatgctgggtattgtgatggcggtgctgactggcctgatgctcaagtacaccatcatgcgcggtgaagcgacgccgtttgtcatggagttgccggtctatcatgtaccacacgttaaaagcctgattatccagacctggcagcgtctgaaaggcttcgttctgcgtgctggtaaagtgatcatcatcgtcagcattttcctgagcgctttcaacagcttctcgctgagcgggaaaatcgtcgataacatcaacgactcggcgctggcgtccgtcagccgggtgatcaccccggtcttcaagccaattggcgtgcatgaagataactggcaggcaacggttggcctgtttacaggtgccatggcgaaagaagtggtagtgggtacgctcaacaccctctacaccgcagaaaatattcaggacgaagagttcaatccggcagaatttaacctcggtgaagagctgttcagtgcgatagatgaaacctggcagagcctgaaagacaccttcagccttagcgtactgatgaaccccattgaagccagcaaaggcgacggcgaaatgggtaccggggcgatgggcgtgatggatcagaaattcggtagcgcagcagcagcttacagctacctgattttcgtcctgctgtatgtaccatgtatctcggtgatgggggctatcgcccgtgaatcaagccgtggctggatgggcttctccatcctgtgggggctgaatatcgcttactcactggcaacattgttctatcaggtcgccagctacagtcagcatccaacttacagcctggtgtgcattctggcggttatcctgtttaacatcgtggttatcggtctgctgcgccgcgcgcgtagccgggtggatatcgaactgctggcaacccgcaagtcggtaagcagttgctgcgcagccagcaccaccggtgattgccattaa

SEQ ID NO: 21 - DNA, 6916 bp, Artificial Sequence
  Synthetic
  gene (1)..(6916): Plasmid (pPS16)
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagagccactacctcggcacgattaagagcacagtgctgccataagacgtccattgctttaactaagtgatcgatcatcccagagtcgtgaacaggtgaaggagtgaaacgcaagcgctcggttccacgaggaactgtcgggaagttgataggctgtacataaatgccgaagtgttccagcagcatgtcagaaatcatcttgcagtggaccggatcgccgacgtgtacagggacgatatgagacccgtgatcaataataggcagacccaatcccttcaaacgcatctttaaaatacgcgcctgtgtctggtgcttctcacgtaattctacgtcacccttcaggtggcgaacggaagcagcggccccagcggcgaccaccggcggcagagaagtggagaaaataaatcccggagcatatgagcgtaccgcatcacacatcttagaagaggcggcaatataccctccgaaaactccgtaggctttacccagcgtgccgttaataatgtcgatgcggtccatcagtccgtcacgttcagcaacaccaccaccgcgcggtccatacattcccaccgcgtgtacttcatcaatgtactttaatgctccaaattcatctgcgatgtcgcaaatttcctcaatgcgtccgaagtcaccgtccatactatatacactctcgaacgcaaccaaaattgggcgatccttcccaatgctggtaagaatgcggcgcaaatcatccaggtcattatgtttgaagatgtgtttttcagttcccgagcggcggatcccttcgatcatactagcatgattcagcttgtcggagacaatgacaagtccgggaataagttgaggcaacgtagacaacgtagcgtcgtttgcgatgtatgctgagctaaagactaacgctgcttccttcccatgtaaatctgccagttctgcttccagacgtttatgatataatgttgtgccggagatattgcgggtacccccagaccccgcacctgtcgagtcaagtgcctcgtgcattgctccaagaactaccgggtgctgtcccatccccaagtaatcgttgccgcaccagaccgtaatctctttctcagatccatccggcttacgccacatagccttcgggaaagcgcccttgcggcgttcgatgtcgatgaatgtgcgataacgcccttcagtgtgtaaacggttcaatgcagtatctaatgccaagttataatccatggtatatctccttcttaaagtctcgagctatcggtttaaaacatcatttcgatgggatagagccccgacaatcgccccaacttcgtcaattaaattttgttcgatacctaactctttgagagtattgatcaaattttctgcgatcgcatcaaaatgaacatccgttaaacccatattatctactaaatgtttatgggattctcgcatatctctaccagaaaaagtatcaggacctccaaaagcataagtcataaaagccttttgatgagctttttgtttcttcatatccgtattggcaaagaaataattaactcggtcatcctttaacaccttatcatagaacttatcgactgcaatatctacagcttctttaccgcctaatttttcgtacaaactactcatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 22 - DNA, 7257 bp, Artificial Sequence
  Synthetic
  gene (1)..(7257): Plasmid (pPS23)
ctagatctacttctaaactgaaacaaatttgagggtaggcttcattgtctgcccttatttttttatttaggaaaagtgaacagactaaagagtgttggctctattgctttgagtatgtaaattaggcgttgctgaattaaggtatgatttttgacccctgcaggatcatcttgctgggtacatcaaaatacccaaatccttactctcccctcaatatacaacaaaaaaaactttttgattcactttagtcataaaaattagaatttatctaccgaaatattacataaatgtaatgtatatattttctgatttattccgtgtgagccatgattcataatttataattcataatttctaaatatgcccctacaatggatatagaatgtcattttaattataggtatcataatcgtggtagttactccggaaaaaactattgaatcaaattcagtctcacctgctacagatagagtagccgttattcttatgggatatggagaggtggaaagttatgatgattttgccaactataatgaacaagccttaaacctacttacggcaaaatttgcccctgtgccaacatggttatatcccccccttgctaaattgttagcggttttcgatttacatgaatggagtcatcaacacggcaattttgtttctcctcataatgctattttcgagcgtcaaagagcaaaaattgagcatcagttgcaacacacttggggagaaaaagtcaaagtatttaaggctttcaacttctgtaaacctcatttaccctatcaagttttagagcaggttaaagctgaaggatttgagaaaattctcatttatcccctcttagtagttgattctatcttcaccagtggaattgcgatcgagcaaataaacgaggcgttagcacaaaaagacaaaaaagaagaccattgggttaaagccttacgttatataccttctttctacaataaaccagagtatatcgatttattagctaaattagtagaagaagaaatcgacgcaaaattaagcaatgcttatttaccttcagaaattggtattgttttaatgaatcacggttgccctcatgaagcaaaaggatttacttcgggtattgatgaaagtcaagccttatatgaattggtaagagaaaaactcatttatcgttatcccttaatttccgtaggttggctcaaccatcaaacccctctaattaaatggactcaacctaatgcggagttagcaggaagaaacttgattgagttaggggcaaaagccttaatatttatgcccattggctttgcaacggaaaaccatgaaaccttgttagatgtagaacacattattcaagcattatacgttaaatatcccgatgtcaattatgtccagatggaatgcgttaacgataatgaagaattttgtgctatggcggcacaatgggttaatcctttgattgaatccttattaacggaagaaggggcgcgccacctgcaggcatgcaagcttggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcttgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggaaattcgagctcaaggcccaggcggccgcgtatgtgaattcctgatttggttgacgtagtttttgaatcgcttttgcctgaatttgtctaactctttcccttgataaattcataattgaggcagtatcactaagggaataaattttgccattttcaaacccataacgcagtctcagaattttttgttctctttcattgagtaaagaaagcatagagcgcaaatccttttgcatagatactctcattaagttttcttcgggagttgcagattctgtttcaattaattccactaattcagtgttataatcttctcccactttcatttctaaggatatggagcggggtatttgatttaaaaattgacgcaaatcaagggaagtcatatccatttctttggctatttcatctactgtggcgattctgcccaatttttgggataattgtctctgtactttttttagttgatttaatctttcagtaatatggataggaagacgaatggttctagcttgagttgcgatcgctcttgtcattccctgacgaatccaccaataagcataagtgctgaaatgatagcctttggtatgatcaaatttttcaacggctttttctaaaccgagagtaccttcttgaattaagtctaataattctaatcctctattttgatattttttggcaatagagacaactaaacgcaaattagcattaatcattttctgttttgctttaatgccattagccaaaatagtttctaactctgatagtttgacttgtgctaaattagcccatgtttcttttccttctgtaattatcgattctacttttttcggagttaaattgagttgttgagcgatttttcctattttgactgcttcagagtgattcgctaccaaagaatcaaatattgtcatgatttccaaatattttgcaagaactggcagttccctatttttgtctctgattgccaataaattttgagaggccgtacttcgacaagacctggaaaagcggaatattgtcaaccagttgttaacactttcccacccttccaggccagctagactagtttattttcctacaacttttgtaatctcccccttcacgtagtgaacaaattcttctaactgatctgctctagaagctaacctatcttcctcttgtcctaaataagcttgacgtgcttccaaaattacgggttgatattgagcaggtaatcgttccatggcccaatcggctgccacgtctttaggagcaattttccctgtaactgcagaataccaaatacgagataaagtaagtacaacatttcgttcatcacctgcccagtcggggggactattccataaggttaaagtttcatttaaagcttcgaataagtcttgctcagggacagggtcaaataactcttcggcagcagggccaactaaggccacgctgtgttctctggctttggtaagaagaatcgctaaatcaatatcaatagtggcaggttcaaaaattcctgctaaaatatcattcctttgccattctccaaattgtaactcacgcttcgctggatatctccagggaataatatcgtcatgtactactatggtcacttcaactgcccgaagaatctcagattccccaggggatgcacttgtttctaacaaatcattaattaaggcacgacgagttgtttcatccaggcgaacggtgacagttacaagtaaatctatgtcactgtgaggttttaatcctccatctacagcagatccatataaatggacagctaataaggtgggttctaaatgacgttcgattactcctacaacttcagagagttgtgtgctaacctcggcaataacggcttcacgcattattttctccttttttcgggttaatcctccatctaaggttgtttaatttctttggctagaaacacccgatgccttagatgtgcgaattttgacaacttttctagtatatcattgtcttttttttttttccagatttaccccttgttttattttcttgatggaagttaagatctttttagggtcatctcatttaacagttagttatagggaaaattggcgaggtgatctgtcgacaattaataacttcttcctgtacgggcgaatggccatttgctcctaactaactccgtactgctttgcggaacgagcgtagcgaactctccgaattactaagccttcatccctgatagatgcaaaaaacgaattaaaattatgtgtaaaaagaaaatgtgtctttatttagtagtcaaagttacaaaatattaagaatcaaattaataatgtattgggcagttaagtatataagtctttaaatatttatttgtattcaatatattaaggaggatcagccttatggattataatttagctttagatactgctttaaatagattacatactgaaggtagatatagaacttttattgatattgaaagaagaaaaggtgcttttcctaaagctatgtggagaaaacctgatggttctgaaaaagaaattactgtttggtgtggtaatgattatttaggtatgggtcaacatcctgttgttttaggtgctatgcatgaagctttagattctactggtgctggttctggtggtactagaaatatttctggtactactttatatcataaaagattagaagctgaattagctgatttacatggtaaagaagctgctttagttttttcttctgcttatattgctaatgatgctactttatctactttacctcaattaattcctggtttagttattgtttctgataaattaaatcatgcttctatgattgaaggtattagaagatctggtactgaaaaacatatttttaaacataatgatttagatgatttaagaagaattttaacttctattggtaaagatagacctattttagttgcttttgaatctgtttattctatggatggtgattttggtagaattgaagaaatttgtgatattgctgatgaatttggtgctttaaaatatattgatgaagttcatgctgttggtatgtatggtcctagaggtggtggtgttgctgaaagagatggtttaatggatagaattgatattattaatggtactttaggtaaagcttatggtgtttttggtggttatattgctgcttcttctaaaatgtgtgatgctgttagatcttatgctcctggttttattttttctacttctttacctcctgttgttgctgctggtgctgctgcttctgttagacatttaaaaggtgatgttgaattaagagaaaaacatcaaactcaagctagaattttaaaaatgagattaaaaggtttaggtttacctattattgatcatggttctcatattgttcctgttcatgttggtgatcctgttcattgtaaaatgatttctgatatgttattagaacattttggtatttatgttcaacctattaattttcctactgttcctagaggtactgaaagattaagatttactccttctcctgttcatgattctggtatgattgatcatttagttaaagctatggatgttttatggcaacattgtgctttaaatagagctgaagttgttgctcatcatcatcatcatcattga

SEQ ID NO: 23 - DNA, 9244 bp, Artificial Sequence
  Synthetic
  gene (1)..(9244): Plasmid (pPSRR24)
atccggatatagttcctcctttcagcaaaaaacccctcaagacccgtttagaggccccaaggggttatgctagttattgctcagcggtggcagcagccaactcagcttcctttcgggctttgttagcagccggatcgaattcttaatggcaatcaccggtggtgctggctgcgcagcaactgcttaccgacttgcgggttgccagcagttcgatatccacccggctacgcgcgcggcgcagcagaccgataaccacgatgttaaacaggataaccgccagaatgcacaccaggctgtaagttggatgctgactgtagctggcgacctgatagaacaatgttgccagtgagtaagcgatattcagcccccacaggatggagaagcccatccagccacggcttgattcacgggcgatagcccccatcaccgagatacatggtacatacagcaggacgaaaatcaggtagctgtaagctgctgctgcgctaccgaatttctgatccatcacgcccatcgccccggtacccatttcgccgtcgcctttgctggcttcaatggggttcatcagtacgctaaggctgaaggtgtctttcaggctctgccaggtttcatctatcgcactgaacagctcttcaccgaggttaaattctgccggattgaactcttcgtcctgaatattttctgcggtgtagagggtgttgagcgtacccactaccacttctttcgccatggcacctgtaaacaggccaaccgttgcctgccagttatcttcatgcacgccaattggcttgaagaccggggtgatcacccggctgacggacgccagcgccgagtcgttgatgttatcgacgattttcccgctcagcgagaagctgttgaaagcgctcaggaaaatgctgacgatgatgatcactttaccagcacgcagaacgaagcctttcagacgctgccaggtctggataatcaggcttttaacgtgtggtacatgatagaccggcaactccatgacaaacggcgtcgcttcaccgcgcatgatggtgtacttgagcatcaggccagtcagcaccgccatcacaatacccagcatatacagcgagaagaccgccagcgcaccgttctgcccgaagaaggcagccgcgaatactgcgaagatagccagacgcgcgccgcaggacataaacggtgccatcatgatggtcatcagacgttcacgcggtgcatcaagcgtacgtgcacccattaccgacggtacgttacaaccgaaaccgacgatcagcggcacaaaggatttccccggcaagcccagcgcctgcatcagacggtccatcacaaacgccgcacgcgccatatacccggagtcctcaaggaaggagaggaacaggtacatcatgccaatctgcggcaccagtggcagcacggtgttaatgccgccacccaggccctgggcgaggaagatagtcagccagtccgggaagtggagcgtgtagccaatccattgaataccatgcacaaatagcgccacggagccgacgtcaaacagcggctgtaacgccccgccgatgttgatagccagcaggaacatcaggtacatcacaaagaggaaaatcggcagaccgaggaaacggttgagcacgattttatctaccgcagtggtgaaacggctgggttctgccgtcagggtgttgcttaccacatcacagatggcagcaatgcactggtaacgcgcatcggcaatgtgcagcgccggatcgtccatctcattacgcagacgggcgagggcggcatccagatgctgcgacgcttcaccggcgtaggcgcggctgtagatatcgccttccagcatttgcaggcccagccagcgacgttgtttcagcgggatgtcggaaggcatcacttttgccagtgaatctgcttcgttgagcagcggctgtgcgtaatgcaccagttccacattctcgttagctttatagcgatcaatcgccagcttgagcgcttcaataccgcgaccacgggttgaaaccagcgggatcaccggacagcccagacgcgccgacagagcatcaatttcaatacgaatattttgcttctcggcaatgtcgagcatgttcagtgccacaatgcagggaatgccgagttccagcagttgtagcgtcaggtacaggttacgctcaaggttagacgcatccaccacgttaatcagcaggtcggcgtcgccactcaaaatgtagtgacaggcgatttgctcatcgagcgaggtctgcgatgagatggtggtcagagaataggtgccgggcaggtccaccagcgtgacctgatgatcggtggtggagaattgcccttctttacgttcgacggtaacgccagcccagttacctacacgctgacgtgagccagtgagctggttaaataacgttgtcttgccagaatttggattaccaattaagccaatggttaattttttcatggtatatctcctaagctttcactcgagagccactacctcggcacgattaagagcacagtgctgccataagacgtccattgctttaactaagtgatcgatcatcccagagtcgtgaacaggtgaaggagtgaaacgcaagcgctcggttccacgaggaactgtcgggaagttgataggctgtacataaatgccgaagtgttccagcagcatgtcagaaatcatcttgcagtggaccggatcgccgacgtgtacagggacgatatgagacccgtgatcaataataggcagacccaatcccttcaaacgcatctttaaaatacgcgcctgtgtctggtgcttctcacgtaattctacgtcacccttcaggtggcgaacggaagcagcggccccagcggcgaccaccggcggcagagaagtggagaaaataaatcccggagcatatgagcgtaccgcatcacacatcttagaagaggcggcaatataccctccgaaaactccgtaggctttacccagcgtgccgttaataatgtcgatgcggtccatcagtccgtcacgttcagcaacaccaccaccgcgcggtccatacattcccaccgcgtgtacttcatcaatgtactttaatgctccaaattcatctgcgatgtcgcaaatttcctcaatgcgtccgaagtcaccgtccatactatatacactctcgaacgcaaccaaaattgggcgatccttcccaatgctggtaagaatgcggcgcaaatcatccaggtcattatgtttgaagatgtgtttttcagttcccgagcggcggatcccttcgatcatactagcatgattcagcttgtcggagacaatgacaagtccgggaataagttgaggcaacgtagacaacgtagcgtcgtttgcgatgtatgctgagctaaagactaacgctgcttccttcccatgtaaatctgccagttctgcttccagacgtttatgatataatgttgtgccggagatattgcgggtacccccagaccccgcacctgtcgagtcaagtgcctcgtgcattgctccaagaactaccgggtgctgtcccatccccaagtaatcgttgccgcaccagaccgtaatctctttctcagatccatccggcttacgccacatagccttcgggaaagcgcccttgcggcgttcgatgtcgatgaatgtgcgataacgcccttcagtgtgtaaacggttcaatgcagtatctaatgccaagttataatccatggtatatctccttcttaaagtctcgagctatcggtttaaaacatcatttcgatgggatagagccccgacaatcgccccaacttcgtcaattaaattttgttcgatacctaactctttgagagtattgatcaaattttctgcgatcgcatcaaaatgaacatccgttaaacccatattatctactaaatgtttatgggattctcgcatatctctaccagaaaaagtatcaggacctccaaaagcataagtcataaaagccttttgatgagctttttgtttcttcatatccgtattggcaaagaaataattaactcggtcatcctttaacaccttatcatagaacttatcgactgcaatatctacagcttctttaccgcctaatttttcgtacaaactactcatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

(19) United States
(12) Patent Application Publication
(10) Pub. No.: US 2026/0078386 A1
(43) Pub. Date: Mar. 19, 2026

(54) GENETICALLY MODIFIED MICROORGANISM, METHOD(S) AND APPLICATION(S) THEREOF

(71) Applicant: RELIANCE INDUSTRIES LIMITED, Mumbai Maharashtra, (IN)

(72) Inventors: Chitranshu KUMAR, Bangalore (IN); Badrish Ranjitlal SONI, Navsari (IN); Mansi Pankaj VORA, Mumbai, Maharashtra (IN); Sudip Kumar SAMANTA, Hyderabad (IN); Piyush SETHIA, Mumbai (IN)

(21) Appl. No.: 18/562,620

(22) PCT Filed: Jun. 24, 2022
(86) PCT No.: PCT/IB2022/055859

§ 371 Date: Nov. 20, 2023

(30) Foreign Application Priority Data
Jun. 24, 2021 (IN) 202121012887

(51) Int. Cl. C12N 15/74; C07K 14/195; C07K 14/245; C07K 14/805; C12N 9/00; C12N 9/02; C12N 9/48; C12N 9/88; C12N 9/90; C12N 15/70; C12R 1/19

(52) U.S. Cl. CPC C12N 15/74; C07K 14/195; C07K 14/245; C07K 14/805; C12N 9/0008; C12N 9/485; C12N 9/88; C12N 9/90; C12N 9/93; C12N 15/70; C12R 2001/19; C12Y 102/0107; C12Y 304/15006; C12Y 504/03008; C12Y 603/02029; C12Y 603/0203

(57) ABSTRACT

The present disclosure relates to genetically modified bacteria, wherein said genetically modified bacteria is designed to express or overexpress a heme-containing protein. Said protein is optionally expressed or overexpressed in combination with one or more enzymes that catalyze the heme synthesis pathway. Said genetically modified organism allows scalable production of heme-containing protein with the advantage that the expressed protein remains in soluble form without forming inclusion bodies.

### CROSS REFERENCE TO RELATED APPLICATIONS

The present application claims priority under 35 U.S.C. § 371 to International Patent Application No. PCT/IB2022/055859, filed Jun. 24, 2022, which claims priority to and the benefit of Indian Patent Application number 202121012887, filed Jun. 24, 2021. The contents of these applications are hereby incorporated by reference in their entireties.

### SEQUENCE LISTING

The instant application contains a Sequence Listing which has been submitted electronically in ASCII format and is hereby incorporated by reference in its entirety. Said ASCII copy, created on Aug. 21, 2025, is named 056859-0331_SL.txt and is 88,964 bytes in size.

### TECHNICAL FIELD

The present disclosure relates to the fields of biotechnology, microbiology and genetic engineering. In particular, the present disclosure relates to a genetically modified bacteria, wherein said genetically modified bacteria is designed to express or overexpress a heme-containing protein.

### BACKGROUND

With vegetarianism gaining popularity, non-animal derived meat-like material, that resembles red meat in taste and appearance appears to be a product that may be of interest to the vegetarian consumer base. At present, meat substitute food products are prepared, commonly, from the combination of animal protein and vegetable protein. The acceptance of these products by consumers is directly related to the organoleptic qualities such as taste and appearance.

However, many vegetarian meat-like products typically lack taste and color. It is desired for the substitute to resemble an actual meat product. For example, a vegetarian-based ground meat or sausage meat that is similar to real meat would be desirable. Modified Cyanobacteria can be used to produce many types of proteins and other products in an environmentally friendly manner. Said Cyanobacteria and proteins produced therefrom find application in the preparation of meat-like products. However, such products fail to replicate the colour and sometimes the taste of meat. Further problems relate to the scalability of the production, posing limitations on commercial scale production of these proteins.

Another issue is the shrinkage in availability of agricultural land and resources such as water needed for irrigation. Accordingly, given said scenario, also desired are alternative means of meeting the growing nutritional requirements in a manner that is conducive for large scale production while reducing load on cultivation compatible fertile lands and competition with edible crops.

Therefore, the need of the hour is a meat substitute that closely resembles meat in taste, appearance and texture, wherein the production of said substitute may be achieved without increasing burden on agricultural land. Therefore, what is currently required is means to make it feasible to produce such meat substitutes on a large scale.

### SUMMARY OF THE DISCLOSURE

The present disclosure provides a genetically modified bacterium expressing heme-containing protein comprising polynucleotide(s) encoding protein(s) selected from a group comprising HemA, HemL, HemH and FeoB or any combination thereof.

In some embodiments, the genetically modified bacterium is selected from E. coli and Cyanobacterium.

In some embodiments, the heme containing protein is an endogenous heme-containing protein or an exogenous heme-containing protein.

In some embodiments, the heme-containing protein is selected from a group comprising hemoglobin, cyanoglobin, and leghemoglobin.

In some embodiments, the heme-containing protein is endogenous or exogenous cyanoglobin.

In some embodiments, when the genetically modified bacterium is cyanobacterium, it is engineered to knockout Cyanophycin synthetase (CphA) and Cyanophycinase (CphB) proteins.

In some embodiments, the polynucleotide(s) encoding protein(s) selected from a group comprising exogenous cyanoglobin, HemA, HemL, HemH and FeoB or any combination thereof are each under the control of the same or different promoter(s) selected from a group comprising PpilA, PT7, PnirA, Cpcb171 and PcpcB or any combination thereof.

Further provided herein is a method of producing the genetically modified bacterium as described above, comprising—
a) Cloning genes encoding protein(s) selected from a group comprising HemA, HemL, HemH and FeoB or any combination thereof into recombinant DNA vector(s);
b) Transforming the recombinant DNA vector(s) into a bacterium expressing heme-containing protein, to obtain the genetically modified bacterium.

In some embodiments, the bacterium expressing heme-containing protein expresses an endogenous heme-containing protein or is genetically modified to express an exogenous heme-containing protein.

In some embodiments, when the bacterium is genetically modified to express an exogenous heme-containing protein, the method comprises
a) Cloning genes encoding the exogenous heme-containing protein and protein(s) selected from a group comprising HemA, HemL, HemH and FeoB or any combination thereof into recombinant DNA vector(s);
b) Transforming the recombinant DNA vector(s) into the bacterium, to obtain the genetically modified bacterium.

In some embodiments, the bacterium expressing heme-containing protein is engineered to knockout Cyanophycin synthetase (CphA) and Cyanophycinase (CphB).

Further provided herein is a method for producing a heme-containing protein comprising
culturing the genetically modified bacterium defined above in a culture medium under
conditions that allow production of the heme-containing protein.

In some embodiments, the heme-containing protein is an endogenous heme-containing protein, the genetically modified bacterium is triggered to overexpress the endogenous heme-containing protein.

In some embodiments, the genetically modified bacteria is cultured in presence of aeration. In some embodiments, the genetically modified bacteria is cultured in presence of a CO₂/air mixture of about 1% to about 4% (v/v) CO₂ in air. In some embodiments, the genetically modified bacteria is cultured at a temperature from about 15° C. to about 40° C. In some embodiments, the genetically modified bacteria is cultured for a time-period ranging from about 5 hours to about 10 days. In some embodiments, the genetically modified bacteria is cultured at a relative humidity of about 60% to about 80%. In some embodiments, the genetically modified bacteria is cultured at a pH of about 6 to about 8. In some embodiments, the genetically modified bacteria is cultured in batch mode, fed-batch mode, semi-turbidostatic mode, or any combination thereof.

In some embodiments, the produced heme-containing protein is subjected to purification; and wherein the purification is performed by method(s) selected from a group comprising chromatography, heating, DCM method, PEG method and dual aqueous phase extraction or any combination thereof.

Further provided herein is the use of the genetically modified bacterium as defined above or the heme-containing protein produced therefrom to prepare a pharmaceutical or food product.

In some embodiments provided herein is a a pharmaceutical or food product comprising the genetically modified bacterium as defined above or the heme-containing protein produced therefrom.

Addressing the aforesaid need in the art, provided herein is a genetically modified microorganism comprising polynucleotide(s) encoding protein(s) selected from a group comprising heme-containing protein and enzyme(s) that catalyze the heme synthesis pathway or any combination thereof.

### BRIEF DESCRIPTION OF THE ACCOMPANYING FIGURES

FIG. 1 depicts the heme synthesis pathway in Cyanobacteria.

FIG. 2 depicts the map of dual expression vector pPS16 for expression of Cyanoglobin and HemA in E. coli.

FIG. 3 (a) depicts that overexpression of Cyanoglobin with HemA turns E. coli cells red on plate as well as in media at low agitation (b) Expression of Cyanoglobin turned E. coli cells red.

FIG. 4 depicts a map of recombinant integrative vector pPS23 for expression of HemA in Cyanobacteria.

FIG. 5 depicts expression vector for Cyanoglobin expression in Cyanobacteria.

FIG. 6 depicts exponential phase of growth of E. coli cells in TB media for Cyanoglobin induction.

FIG. 7 depicts uninduced (TB_UI) and induced (TB_I) E. coli cell pellet. The induced cell pellet at the optimum induction OD is brick red in color.

FIG. 8 depicts chromatograph of affinity run for Cyanoglobin expressed in E. coli.

FIG. 9 depicts results of Affinity Chromatography-SDS PAGE analysis for Cyanoglobin expressed in E. coli.

FIG. 10 depicts ion exchange Chromatograph run for Cyanoglobin expressed in E. coli.

FIG. 11 depicts SDS PAGE Reducing Gel of Non chromatographic method for Cyanoglobin expressed in E. coli.

FIG. 12 depicts separation of three layers using DCM method with top layer showing the Cyanoglobin fraction extracted from homogenized and unhomogenized E. coli cells.

FIG. 13 depicts PAGE gel for extracted sample in the top layer obtained in the DCM method to check the purity of the sample; ‘L’ refers to the ladder. ‘E. coli lysate’ is the cell free extract obtained after homogenizing E. coli cells.

FIG. 14 depicts results of the RNA quality check by gel electrophoresis in Expression analysis of E. coli strains producing Cyanoglobin, Lane M: 1 Kb plus DNA ladder, Lane C: Empty vector control, Lane C2, C3, C4: recombinant clones having Cyanoglobin overexpressed.

FIG. 15 depicts PCR for housekeeping gene used was RPOS (202 bp), (B): PCR for Cyanoglobin gene. Lane C1 to C5: recombinant clones having Cyanoglobin gene (375 bp); Lane C: empty vector control; Lane N: NTC (no template control).

FIG. 16 depicts semi quantitative amplification curves of gene expression of housekeeping (RPOS) gene and gene of interest (GOI) in recombinant E. coli clones having Cyanoglobin gene (375 bp).

FIG. 17 depicts A) Coomassie stained SDS PAGE of with respective controls, Lane M: Precision Plus Protein™ Dual Color Standards, Lane C: empty vector control, Lane C2-C4: E. coli induced clones harboring Cyanoglobin. B) Western blot analysis of Cyanoglobin with respective controls, Lane M: Precision Plus Protein™ Dual Color Standards, Lane C: empty vector control, Lane C2-C4: E. coli induced clones expressing Cyanoglobin protein of molecular size 14 KD.

FIG. 18 depicts RNA quality checked by gel electrophoresis, Lane C1 to C5: Recombinant clone at OD1, OD2, OD3, OD4, OD6 during gene expression analysis in Cyanobacteria.

FIG. 19 depicts semi-quantitative gene expression of Cyanoglobin in recombinant Cyanobacteria strains at different growth stages, A: PCR for housekeeping gene RPOS (202 bp), B: PCR for Cyanoglobin gene. Lane 1 to 6: recombinant clones at different growth stages OD 1 to OD 6 having Cyanoglobin gene (192 bp); Lane W: wild type strain; Lane N: NTC (no template control).

FIG. 20 depicts gene expression comparison in recombinant Cyanobacteria clone at different growth stages.

FIG. 21 depicts western blot analysis of Cyanoglobin gene under NirA promoter, Lane 1: Prestained ladder, Lane 2: Blank, Lane 3: wild type strain, Lanes 4 and 5: clones having Cyanoglobin gene under NirA promoter at Sig-C neutral site.

FIG. 22 depicts western blot analysis of recombinant clones having Cyanoglobin gene under NirA promoter at two different neutral sites. Lane 1: Prestained ladder, Lane 2: wild type strain, Lane 3: Blank, Lane 4 to 7: recombinant clones having gene under NirA at Sig-C site, Lane: 8 clone having Cyanoglobin gene under NirA at PsyB site.

FIG. 23 depicts western blot analysis of recombinant clones having Cyanoglobin gene under Cpcb171 promoter. Lane 1: Prestained ladder, Lane 2 to 7: recombinant clones having gene under Cpcb 171 promoter, Lane 8: wild type strain.

FIG. 24 depicts (A) simulated growth conditions; Light intensity in orange, Measured Temperature in yellow and pH in green, (B) PBR L7, L8 with clone having Cyanoglobin gene under strong promoter CpcB171 at Sig-C site, PBR L10 with wild type strain.

FIG. 25 depicts western blot analysis of clone having Cyanoglobin gene under strong promoter CpcB171 at Sig-C site under outdoor stimulated condition. Lane 1: Prestained ladder, Lane 2 to 5: clone from PBR L7 at OD 2, OD 4, OD 6 OD 8, Lane 6 to 9: clone from PBR L8 at OD 2, OD 4, OD 6 OD 8, Lane 10: wild type strain.

FIG. 26 depicts pI determination of affinity chromatography purified cyanoglobin. A) depicts pH3-pH10 gradient strip with well used to locate cyanoglobin based on iso-electric point. B) Fractions loaded on SDS-PAGE to locate cyanoglobin, showing two forms (oxygenated and deoxygenated) at pI 6.6 and 7.2.

FIG. 27 depicts diagnostic PCR for confirmation of FeoB positive clones by restriction enzyme double digestion using EcoRI and BamHI in E. coli transformation experiments using plasmid pET28a-Cyanoglobin-hemARs-FeoB.

### DETAILED DESCRIPTION OF THE INVENTION

Unless otherwise defined herein, scientific and technical terms used in connection with the present invention shall have the meanings that are commonly understood by those of ordinary skill in the art. Generally, nomenclatures used in connection with, and techniques of, biochemistry, enzymology, molecular and cellular biology, microbiology, genetics and protein and nucleic acid chemistry and hybridization described herein are those well-known and commonly used in the art. The following terms, unless otherwise indicated, shall be understood to have the following meanings:

The term “vector” as used herein is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

The term “Cyanobacterium” or “Cyanobacteria” refers to a member from the group of photoautotrophic prokaryotic microorganisms which can utilize solar energy and fix carbon dioxide. Cyanobacteria are also referred to as blue-green algae.

As used herein, the term “genetically modified” refers to any change in the endogenous genome of a wild type cell or to the addition of non-endogenous genetic code to a wild type cell, e.g., the introduction of a heterologous gene. More specifically, such changes are made by the hand of man through the use of recombinant DNA technology or mutagenesis. The changes can involve protein coding sequences or non-protein coding sequences, including regulatory sequences such as promoters or enhancers.

The terms “polynucleotide” and “nucleic acid” also refer to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs. It will be understood that, where required by context, when a nucleotide sequence is represented by a DNA sequence (i.e., A, T, G, C), this also includes an RNA sequence (i.e., A, U, G, C) in which “U” replaces “T.” The nucleic acids of this present invention may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages, charged linkages, alkylators, intercalators, pendent moieties, modified linkages, and chelators. Also included are synthetic molecules that mimic polynucleotides in their ability to bind to a designated sequence via hydrogen bonding and other chemical interactions.

The term “nucleic acid” (also referred to as polynucleotide) is also intended to include nucleic acid molecules having an open reading frame encoding a polypeptide, and can further include non-coding regulatory sequences and introns. In addition, the terms are intended to include one or more genes that map to a functional locus. In addition, the terms are intended to include a specific gene for a selected purpose. The gene can be endogenous to the host cell or can be recombinantly introduced into the host cell.

In one aspect the invention also provides nucleic acids which are at least 60%, 70%, 80%, 90%, 95%, 99%, or 99.5% identical to the nucleic acids disclosed herein.

The term “endogenous gene” refers to a native gene in its natural location in the genome of an organism.

As used herein, “exogenous” refers to any nucleic acid sequence that is introduced into a cell from, for example, the same or a different organism or a nucleic acid generated synthetically (e.g., a codon-optimized nucleic acid sequence). For example, an exogenous nucleic acid can be a nucleic acid from one microorganism (e.g., one genus or species of methylotrophic yeast) that is introduced into a different genus or species of methylotrophic yeast. Reference to ‘exogenous heme-containing protein’ or ‘exogenous cyanoglobin’ in the present disclosure, particularly in environments wherein a corresponding endogenous protein is produced (for example, Cyanobacteria which expresses endogenous cyanoglobin) envisages the possibility of action or expression of the exogenous protein over and above or in addition to the action or expression of the corresponding endogenous protein.

A “heterologous” nucleic acid refers to any nucleic acid sequence that is not native to an organism (e.g., a heterologous nucleic acid can be a nucleic acid from one microorganism (e.g., one genus or species of methylotrophic yeast, whether or not it has been codon-optimized) that is introduced into a different genus or species of methylotrophic yeast). “Heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

A “heterologous” protein is a protein encoded by a heterologous nucleic acid.

The term “globin” refers to a type of heme-containing globular protein involved in reversible binding and transporting of oxygen utilizing a heme prosthetic group. Globins incorporate a series of alpha helical segments known as globin folds.

The terms “heme-containing protein” or “heme-binding protein” refer to a polypeptide that can bind to a heme group. Examples of heme-containing proteins include but are not limited to hemoglobin, Cyanoglobin, and leghemoglobin.

The term “heme” refers to a heterocyclic organic compound cofactor made up of four joined pyrrolic groups, having an iron (Fe²⁺) group located in the center of the molecule. The heme iron often acts as an electron source or sink during electron transfer or redox chemistry.

The term “fragment” refers to a nucleotide sequence of reduced length relative to the reference nucleic acid and comprising, over the common portion, a nucleotide sequence substantially identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least about 6, 50, 100, 200, 500, 1,000, to about 1,500 or more consecutive nucleotides of a polynucleotide according to the invention.

The term “expression”, as used herein, refers to the transcription and stable accumulation mRNA derived from a nucleic acid or polynucleotide. Expression may also refer to translation of mRNA into a protein or polypeptide.

An “expression cassette” or “cassette” or “construct” refers to a series of polynucleotide elements that permit transcription of a gene in a host cell. Typically, the expression cassette includes a promoter and a heterologous or native polynucleotide sequence that is transcribed. Expression cassettes or constructs may also include, e.g., transcription termination signals, polyadenylation signals, and enhancer elements.

The term “codon optimization”, “codon optimized” or obvious variants thereof refer to the modification of at least some of the codons present in a heterologous gene sequence from a triplet code that is infrequently used in the host organism to a triplet code that is more common in the particular host organism. This can result in a higher expression level of the gene of interest.

The term “transformation” is used herein to mean the insertion of heterologous genetic material into the host cell. Typically, the genetic material is DNA on a plasmid vector, but other means can also be employed. General transformation methods and selectable markers for bacteria and Cyanobacteria are known in the art (Wirth, Mol Gen Genet. 216:175-177 (1989); Koksharova, Appl Microbiol Biotechnol 58:123-137 (2002). Additionally, transformation methods and selectable markers for use in bacteria are well known (see, e.g., Sambrook et al, supra).

The term “selectable marker” means an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, spectinomycin, kanamycin, zeocin, chloramphenicol, hygromycin, and the like.

A “polypeptide” is a polymeric compound comprised of covalently linked amino acid residues. A “protein” is a polypeptide that performs a structural or functional role in a living cell.

A “promoter” is an array of nucleic acid control sequences that direct transcription of an associated polynucleotide, which may be a heterologous or native polynucleotide. A promoter includes nucleic acid sequences near the start site of transcription, such as a polymerase binding site. The promoter also optionally includes distal enhancer or repressor elements which can be located as much as several thousand base pairs from the start site of transcription. The term “promoter” is intended to include a polynucleotide segment that can transcriptionally control a gene of interest, e.g., a pyruvate decarboxylase gene that it does or does not transcriptionally control in nature. In one embodiment, the transcriptional control of a promoter results in an increase in expression of the gene of interest. In an embodiment, a promoter is placed 5′ to the gene of interest. A heterologous promoter can be used to replace the natural promoter, or can be used in addition to the natural promoter. A promoter can be endogenous with regard to the host cell in which it is used or it can be a heterologous polynucleotide sequence introduced into the host cell, e.g., exogenous with regard to the host cell in which it is used. Promoters of the invention may also be inducible, meaning that certain exogenous stimuli (e.g., nutrient starvation, heat shock, mechanical stress, light exposure, etc.) will induce the promoter leading to the transcription of the gene.

The term “knockout” refers to reduction or suppression of the expression of a protein encoded by an endogenous DNA sequence in a cell.

Except as otherwise noted, all technology used among the present invention and scientific terminology have with the present invention under the identical implication of technical field those of ordinary skill institute common sense. Exemplary method and material are described below, although similar or be equal to the method for the method described among the present invention and material and material also can be used for embodiment of the present invention and it will be apparent to those skilled in the art.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

All publications, patents and other references mentioned herein are hereby incorporated by reference in their entireties.

Reference throughout this specification to “one embodiment” or “an embodiment” means that a particular feature, structure or characteristic described in connection with the embodiment may be included in at least one embodiment of the present disclosure. Thus, the appearances of the phrases “in one embodiment” or “in an embodiment” in various places throughout this specification may not necessarily all refer to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

The term “about” is used herein to mean approximately, in the region of, roughly, or around. When the term “about” is used in conjunction with a numerical value/range, it modifies that value/range by extending the boundaries above and below the numerical value(s) set forth. In general, the term “about” is used herein to modify a numerical value(s) above and below the stated value(s) by a variance of 20%.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The use of the expression ‘at least’ or ‘at least one’ suggests the use of one or more elements or ingredients or quantities, as the use may be in the embodiment of the disclosure to achieve one or more of the desired objects or results. Throughout this specification, the word ‘comprise’, or variations such as ‘comprises’ or ‘comprising’ wherever used, will be understood to imply the inclusion of a stated element, integer or step, or group of elements, integers or steps, but not the exclusion of any other element, integer or step, or group of elements, integers or steps. Numerical ranges stated in the form ‘from x to y’ include the values mentioned and those values that lie within the range of the respective measurement accuracy as known to the skilled person. If several preferred numerical ranges are stated in this form, of course, all the ranges formed by a combination of the different end points are also included.

It is to be noted that the term ‘a’ or ‘an’ entity refers to one or more of that entity. As such, the terms ‘a’ (or ‘an’), ‘one or more’, and ‘at least one’ can be used interchangeably herein.

As regards the embodiments characterized in this specification, it is intended that each embodiment be read independently as well as in combination with another embodiment. For example, in case of an embodiment 1 reciting 3 alternatives A, B and C, an embodiment 2 reciting 3 alternatives D, E and F and an embodiment 3 reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Addressing the problems identified in the art, disclosed herein is a genetically modified microorganism that expresses or overexpresses a heme-containing protein along with one or more enzymes that catalyze the heme synthesis pathway and/or an iron transporter protein.

Particularly provided herein is genetically modified bacteria expressing heme-containing protein comprising polynucleotide(s) encoding protein(s) selected from a group comprising heme-containing protein, HemA, HemL, HemH and FeoB or any combination thereof.

In some embodiments, the genetically modified bacteria expressing heme-containing protein comprises polynucleotide(s) encoding FeoB and protein(s) selected from a group comprising heme-containing protein, HemA, HemL and HemH or any combination thereof.

In some embodiments, the genetically modified bacteria expressing heme-containing protein comprises polynucleotide(s) encoding FeoB, HemA and protein(s) selected from a group comprising heme-containing protein, HemL and HemH or any combination thereof.

In some embodiments, the genetically modified bacteria expressing heme-containing protein comprises polynucleotide(s) encoding FeoB and HemA.

In some embodiments, the genetically modified bacteria is of genus Escherichia and/or Cyanobacteria.

In some embodiments, the bacteria of genus Escherichia is Escherichia coli (E. coli).

In some embodiments, the bacteria of genus Cyanobacteria is selected from a group comprising Cyanobacterium sp., Anabaena sp., Crocosphaera sp., Geitlerinema sp., Geminocystis sp., Leptolyngbya sp., Microcystis sp., Nostoc sp., Nostocaceae sp., Synechococcus sp., Synechocystis sp. and Thermosynechococcus sp.

In some embodiments, the genetically modified microorganism is bacteria selected from a group comprising Escherichia coli (E. coli) and Cyanobacterium aponinum or a combination thereof.

In exemplary embodiments, the genetically modified E. coli is NEB BL21 pLysS or BL21 (DE3).

In some embodiments, the heme containing protein expressed by the genetically modified bacteria is selected from a group comprising Hemoglobin, Cyanoglobin, and Leghemoglobin.

In some embodiments, the heme containing protein expressed by the genetically modified bacteria is an endogenous heme-containing protein or an exogenous heme-containing protein. In a non-limiting example, the heme-containing protein is an endogenous heme-containing protein in case of Cyanobacteria or an exogenous heme-containing protein in case of E. coli. In another non-limiting example, the heme-containing protein is an exogenous heme-containing protein and the genetically modified bacteria is either E. coli or Cyanobacteria.

In some embodiments of the present disclosure, the heme-containing protein is a heterologous heme-containing protein. In non-limiting embodiments, the nucleic acid encoding the heterologous heme-containing protein is derived from microbial, animal or plant sources. Said nucleic acid encoding the heme-containing protein can be a naturally occurring sequence or a synthetic sequence. In case of a naturally occurring sequence, also envisaged herein are variants such as related naturally occurring structural variants, and synthetic non-naturally occurring analogs thereof.

In some embodiments, the heme-containing protein is Cyanoglobin. Reference to genetically modified bacteria expressing Cyanoglobin encompasses genetically modified bacteria having single or multiple copies of the Cyanoglobin gene including or excluding the endogenous Cyanoglobin gene (for instance, in case of Cyanobacteria).

Accordingly, in further preferred embodiments, the present disclosure provides genetically modified bacteria expressing Cyanoglobin comprising polynucleotide(s) encoding protein(s) selected from a group comprising HemA, HemL, HemH and FeoB or any combination thereof.

In some embodiments, the present disclosure provides genetically modified E. coli expressing Cyanoglobin comprising polynucleotide(s) encoding protein(s) selected from a group comprising HemA, HemL, HemH and FeoB or any combination thereof.

In some embodiments, the present disclosure provides genetically modified Cyanobacteria expressing Cyanoglobin comprising polynucleotide(s) encoding protein(s) selected from a group comprising HemA, HemL, HemH and FeoB or any combination thereof.

In some embodiments, the present disclosure provides genetically modified E. coli expressing Cyanoglobin comprising polynucleotide(s) encoding FeoB and protein(s) selected from a group comprising HemA, HemL and HemH or any combination thereof.

In some embodiments, the present disclosure provides genetically modified E. coli expressing Cyanoglobin comprising polynucleotide(s) encoding FeoB and HemA.

In some embodiments, the present disclosure provides genetically modified Cyanobacteria expressing Cyanoglobin comprising polynucleotide(s) encoding FeoB and protein(s) selected from a group comprising HemA, HemL and HemH or any combination thereof.

In some embodiments, the present disclosure provides genetically modified Cyanobacteria expressing Cyanoglobin comprising polynucleotide(s) encoding FeoB and HemA.

HemA, HemL HemH are enzymes that catalyze the heme synthesis pathway in the cell and FeoB is an iron transporter protein. Said proteins enhance the heme content in the cell and therefore allow improved capture of the heme by heme-containing proteins.

In some embodiments, the genetically modified microorganism is genetically modified Cyanobacteria, wherein the genetic modification is expression or overexpression of protein(s) selected from a group comprising HemA, Hem L, HemH and FeoB or any combination thereof, wherein once expressed, said proteins enhance the heme content in the cell, allowing improved capture of the heme by endogenous Cyanoglobin of the Cyanobacteria. When the heme-containing protein is an endogenous protein, for example an endogenous globin protein such as Cyanoglobin, in some embodiments, the genetically modified microorganism is programmed to overexpress the Cyanoglobin.

Accordingly, in some embodiments, the genetically modified microorganism is genetically modified Cyanobacteria, wherein the Cyanobacteria expresses endogenous heme-containing protein and comprises polynucleotide(s) encoding protein(s) selected from a group comprising HemA, HemL, HemH and FeoB or any combination thereof.

In some embodiments, the genetically modified microorganism is genetically modified Cyanobacteria, wherein the Cyanobacteria expresses endogenous heme-containing protein and comprises polynucleotide(s) encoding FeoB and protein(s) selected from a group comprising HemA, HemL and HemH or any combination thereof.

In some embodiments, the genetically modified microorganism is genetically modified Cyanobacteria, wherein the Cyanobacteria expresses endogenous heme-containing protein and comprises polynucleotide(s) encoding FeoB and HemA.

In some embodiments, the genetically modified microorganism is genetically modified Cyanobacteria, wherein the Cyanobacteria expresses endogenous Cyanoglobin and comprises polynucleotide(s) encoding protein(s) selected from a group comprising HemA, HemL, HemH and FeoB or any combination thereof.

In some embodiments, the genetically modified microorganism is genetically modified Cyanobacteria, wherein the Cyanobacteria expresses endogenous Cyanoglobin and comprises polynucleotide(s) encoding FeoB and protein(s) selected from a group comprising HemA, HemL and HemH or any combination thereof.

In some embodiments, the genetically modified microorganism is genetically modified Cyanobacteria, wherein the Cyanobacteria expresses endogenous Cyanoglobin and comprises polynucleotide(s) encoding FeoB and HemA.

In some embodiments, the genetically modified microorganism is genetically modified E. coli, wherein the E. coli expresses exogenous heme-containing protein and comprises polynucleotide(s) encoding protein(s) selected from a group comprising HemA, HemL, HemH and FeoB or any combination thereof. Said exogenous heme-containing protein in some embodiments, is a heterologous heme-containing protein.

In some embodiments, the genetically modified microorganism is genetically modified E. coli, wherein the E. coli expresses exogenous heme-containing protein and comprises polynucleotide(s) encoding FeoB and protein(s) selected from a group comprising HemA, HemL and HemH or any combination thereof.

In some embodiments, the genetically modified microorganism is genetically modified E. coli, wherein the E. coli expresses exogenous heme-containing protein and comprises polynucleotide(s) encoding FeoB and HemA.

In some embodiments, the genetically modified microorganism is genetically modified E. coli, wherein the E. coli expresses exogenous Cyanoglobin and comprises polynucleotide(s) encoding protein(s) selected from a group comprising HemA, HemL, HemH and FeoB or any combination thereof. Said exogenous Cyanoglobin in some embodiments, is a heterologous Cyanoglobin.

In some embodiments, the genetically modified microorganism is genetically modified E. coli, wherein the E. coli expresses exogenous Cyanoglobin and comprises polynucleotide(s) encoding FeoB and protein(s) selected from a group comprising HemA, HemL and HemH or any combination thereof.

In some embodiments, the genetically modified microorganism is genetically modified E. coli, wherein the E. coli expresses exogenous Cyanoglobin and comprises polynucleotide(s) encoding FeoB and HemA.

In some embodiments, the genetically modified microorganism is genetically modified Cyanobacteria, wherein the Cyanobacteria expresses exogenous heme-containing protein and comprises polynucleotide(s) encoding protein(s) selected from a group comprising HemA, HemL, HemH and FeoB or any combination thereof. Said exogenous heme-containing protein in some embodiments, is a heterologous heme-containing protein.

In some embodiments, the genetically modified microorganism is genetically modified Cyanobacteria, wherein the Cyanobacteria expresses exogenous heme-containing protein and comprises polynucleotide(s) encoding FeoB and protein(s) selected from a group comprising HemA, HemL and HemH or any combination thereof.

In some embodiments, the genetically modified microorganism is genetically modified Cyanobacteria, wherein the Cyanobacteria expresses exogenous heme-containing protein and comprises polynucleotide(s) encoding FeoB and HemA.

In some embodiments, the genetically modified microorganism is genetically modified Cyanobacteria, wherein the Cyanobacteria expresses exogenous Cyanoglobin and comprises polynucleotide(s) encoding protein(s) selected from a group comprising HemA, HemL, HemH and FeoB or any combination thereof. Said exogenous Cyanoglobin in some embodiments, is a heterologous Cyanoglobin.

In some embodiments, the genetically modified microorganism is genetically modified Cyanobacteria, wherein the Cyanobacteria expresses exogenous Cyanoglobin and comprises polynucleotide(s) encoding FeoB and protein(s) selected from a group comprising HemA, HemL and HemH or any combination thereof.

In some embodiments, the genetically modified microorganism is genetically modified Cyanobacteria, wherein the Cyanobacteria expresses exogenous Cyanoglobin and comprises polynucleotide(s) encoding FeoB and HemA.

Taken together, the genetically modified bacteria is designed such that the proteins selected from HemA, HemL, HemH, and FeoB or any combination thereof increase the overall pool of Heme inside the cytoplasm which are inturn be available for heme capture by the heme containing protein, wherein the expression of the heme containing protein may also be enhanced by way of introduction of an exogenous gene encoding the same or by triggering overexpression of a endogenous heme containing protein. This therefore leads to higher amount of functional heme containing protein being produced by the cell. Each of HemA, Hem L, HemH, alone or in combination, bear the function of catalyzing the heme synthesis pathway in a cell and are expected to act in the same manner, wherein the effect observed upon expression of anyone, for eg. HemA, in the cell, in combination with a heme containing proteins and optionally FeoB, is extrapolatable to the expression of the other two for eg. Hem L and HemH, individually, or in combination with each other.

In some embodiments, the HemA gene is from Rhodobacter sphaeroides. Expression of HemA increases the flux of heme through heme synthesis pathway, wherein said increase in flux is in turn through enhancement of ALA synthase.

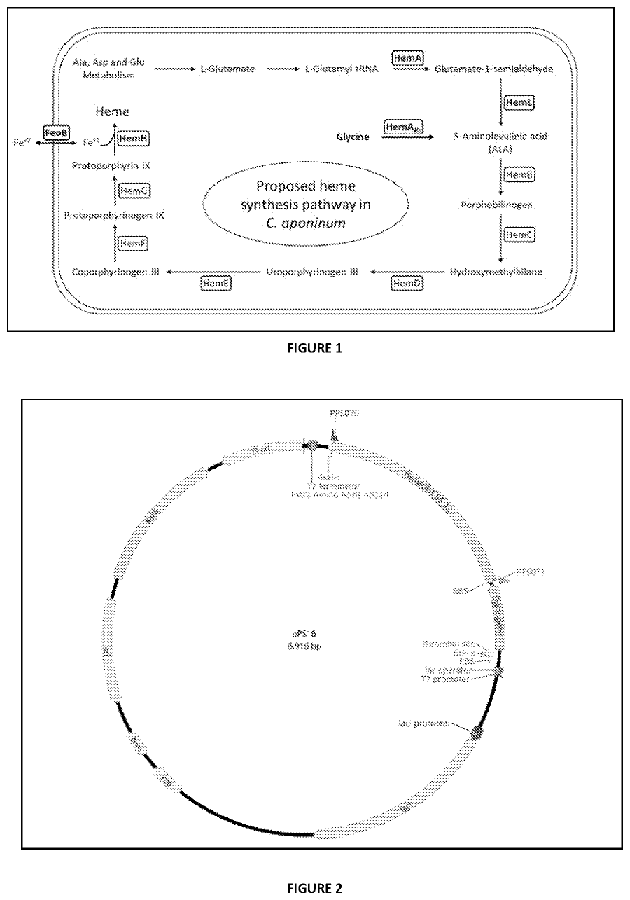

In some embodiments, when the genetically modified microorganism is genetically modified Cyanobacteria, in addition to the aforementioned modifications, the Cyanobacteria may be further engineered to knockout Cyanophycin synthetase (CphA) and Cyanophycinase (CphB) proteins, to achieve increased production of the amino acid selected from a group comprising threonine, alanine, L-proline, lysine, glycine, glutamic acid and combinations thereof. Increased amino acid content, specifically increased content of amino acids such as alanine and glutamic acid allow for enhanced heme synthesis since heme is a product of alanine, aspartic Acid and glutamic acid metabolism in Cyanobacteria (FIG. 1).

In some embodiments, the genetically modified microorganism is genetically modified CphA and CphB knockout Cyanobacteria, wherein the Cyanobacteria expresses endogenous and/or exogenous heme-containing protein and comprises polynucleotide(s) encoding protein(s) selected from a group comprising HemA, HemL, HemH and FeoB or any combination thereof. Said exogenous heme-containing protein in some embodiments, is a heterologous heme-containing protein.

In some embodiments, the genetically modified microorganism is genetically modified CphA and CphB knockout Cyanobacteria, wherein the Cyanobacteria expresses endogenous and/or exogenous heme-containing protein and comprises polynucleotide(s) encoding FeoB and protein(s) selected from a group comprising HemA, HemL and HemH or any combination thereof.

In some embodiments, the genetically modified microorganism is genetically modified CphA and CphB knockout Cyanobacteria, wherein the Cyanobacteria expresses endogenous and/or exogenous heme-containing protein and comprises polynucleotide(s) encoding FeoB and HemA.

In some embodiments, the genetically modified microorganism is genetically modified CphA and CphB knockout Cyanobacteria, wherein the Cyanobacteria expresses endogenous and/or exogenous Cyanoglobin and comprises polynucleotide(s) encoding protein(s) selected from a group comprising HemA, HemL, HemH and FeoB or any combination thereof. Said exogenous Cyanoglobin in some embodiments, is a heterologous Cyanoglobin.

In some embodiments, the genetically modified microorganism is genetically modified CphA and CphB knockout Cyanobacteria, wherein the Cyanobacteria expresses endogenous and/or exogenous Cyanoglobin and comprises polynucleotide(s) encoding FeoB and protein(s) selected from a group comprising HemA, HemL and HemH or any combination thereof.

In some embodiments, the genetically modified microorganism is genetically modified CphA and CphB knockout Cyanobacteria, wherein the Cyanobacteria expresses endogenous and/or exogenous Cyanoglobin and comprises polynucleotide(s) encoding FeoB and HemA.

To summarize the above, the present disclosure provides a genetically modified bacteria comprising nucleotide sequence(s) encoding any combination of proteins selected from the combinations indicated in Table A. In Table A, X represents presence of the nucleotide encoding the specific protein as part of the combination encompassed in each row. Absence of exogenous Cyanoglobin as per the below table is indicative of expression or overexpression of endogenous Cyanoglobin. Accordingly, every single combination provided in Table A represents a separate embodiment of the present disclosure. However, the present disclosure also envisages a merger or mixture of these embodiments to provide for further possible combinations. Thus, for the purposes of the present disclosure, each of the combinations that are derivable from Table A below are envisaged to exist individually, all together or in different combinations within the ambit of the present disclosure.

TABLE A

| | Protein(s) encoded by nucleotide comprised in the | | | | | Further | |
|---|---|---|---|---|---|---|---|
| Genetically | genetically modified microorganism | | | | | modification | |
| modified | Exogenous | | | | | CphA | CphB |
| microorganism | Cyanoglobin | HemA | HemL | HemH | FeoB | KO | KO |
| E. coli | X | | | | X | | |
| E. coli | X | X | | | | | |
| E. coli | X | | X | | | | |
| E. coli | X | | | X | | | |
| E. coli | X | X | X | | | | |
| E. coli | X | X | | X | | | |
| E. coli | X | | X | X | | | |
| E. coli | X | X | X | X | | | |
| E. coli | X | X | | | X | | |
| E. coli | X | | X | | X | | |
| E. coli | X | | | X | X | | |
| E. coli | X | X | X | | X | | |
| E. coli | X | X | | X | X | | |
| E. coli | X | | X | X | X | | |
| E. coli | X | X | X | X | X | | |
| Cyanobacteria | | | | | X | | |
| Cyanobacteria | X | | | | X | | |
| Cyanobacteria | | X | | | | | |
| Cyanobacteria | | | X | | | | |
| Cyanobacteria | | | | X | | | |
| Cyanobacteria | | X | X | | | | |
| Cyanobacteria | | X | | X | | | |
| Cyanobacteria | | | X | X | | | |
| Cyanobacteria | | X | X | X | | | |
| Cyanobacteria | | X | | | X | | |
| Cyanobacteria | | | X | | X | | |
| Cyanobacteria | | | | X | X | | |
| Cyanobacteria | | X | X | | X | | |
| Cyanobacteria | | X | | X | X | | |
| Cyanobacteria | | | X | X | X | | |
| Cyanobacteria | | X | X | X | X | | |
| Cyanobacteria | X | X | | | | | |
| Cyanobacteria | X | | X | | | | |
| Cyanobacteria | X | | | X | | | |
| Cyanobacteria | X | X | X | | | | |
| Cyanobacteria | X | X | | X | | | |
| Cyanobacteria | X | | X | X | | | |
| Cyanobacteria | X | X | X | X | | | |
| Cyanobacteria | X | X | | | X | | |
| Cyanobacteria | X | | X | | X | | |
| Cyanobacteria | X | | | X | X | | |
| Cyanobacteria | X | X | X | | X | | |
| Cyanobacteria | X | X | | X | X | | |
| Cyanobacteria | X | | X | X | X | | |
| Cyanobacteria | X | X | X | X | X | | |
| Cyanobacteria | | | | | X | X | X |
| Cyanobacteria | X | | | | X | X | X |
| Cyanobacteria | | X | | | | X | X |
| Cyanobacteria | | | X | | | X | X |
| Cyanobacteria | | | | X | | X | X |
| Cyanobacteria | | X | X | | | X | X |
| Cyanobacteria | | X | | X | | X | X |
| Cyanobacteria | | | X | X | | X | X |
| Cyanobacteria | | X | X | X | | X | X |
| Cyanobacteria | | X | | | X | X | X |
| Cyanobacteria | | | X | | X | X | X |
| Cyanobacteria | | | | X | X | X | X |
| Cyanobacteria | | X | X | | X | X | X |
| Cyanobacteria | | X | | X | X | X | X |
| Cyanobacteria | | | X | X | X | X | X |
| Cyanobacteria | | X | X | X | X | X | X |
| Cyanobacteria | X | X | | | | X | X |
| Cyanobacteria | X | | X | | | X | X |
| Cyanobacteria | X | | | X | | X | X |
| Cyanobacteria | X | X | X | | | X | X |
| Cyanobacteria | X | X | | X | | X | X |
| Cyanobacteria | X | | X | X | | X | X |
| Cyanobacteria | X | X | X | X | | X | X |
| Cyanobacteria | X | X | | | X | X | X |
| Cyanobacteria | X | | X | | X | X | X |
| Cyanobacteria | X | | | X | X | X | X |
| Cyanobacteria | X | X | X | | X | X | X |
| Cyanobacteria | X | X | | X | X | X | X |
| Cyanobacteria | X | | X | X | X | X | X |
| Cyanobacteria | X | X | X | X | X | X | X |

In some embodiments, the genes of interest encoding any of the aforesaid combination of proteins (exogenous cyanoglobin, HemA, HemL, HemH or FeoB) are inserted into the bacterial chromosome.

In preferred embodiments, the genes of interest encoding any of the aforesaid combination of proteins are not integrated into the bacterial chromosome and are present in an extrachromosomal vector such as but not limited to an extrachromosomal plasmid.

In some non-limiting embodiments, the genes of interest are modified for optimal expression by codon optimization to result in an increased expression of the proteins in the genetically modified bacteria.

In some embodiments, the nucleotide(s) encoding the aforesaid proteins are present on recombinant DNA vectors such as one or more extrachromosomal expression plasmids that are introduced into the bacterial. In some embodiments, the DNA sequences encoding the proteins can be isolated from their respective sources and amplified by polymerase chain reaction (PCR) using specific primers. The amplified PCR fragments may then be digested with the appropriate restriction enzymes and cloned into recombinant DNA vectors such as expression plasmids examples of which include self-replicating plasmid or an integrative plasmid. In an embodiment, the expression plasmid is a heterologous extrachromosomal plasmid pET28a (+).

In some embodiments, the genetically modified bacteria comprise single or multiple copies of each gene of interest encoding protein(s) part of any combination as indicated in Table A.

In some embodiments, the genes encoding any combination of proteins as indicated in Table A are present on one cassette, their expression being under the control of one promoter. In another embodiment, the said genes are carried on separate plasmids under the control of their respective promoters. In some embodiments, genes may be present on a single plasmid in the form of multiple cassettes on one plasmid, each cassette controlled by different individual promoters. The promoters can be one or more of constitutive promoters, regulatable promoters, endogenous promoters and/or heterologous promoters.

In a non-limiting embodiment, the promoter(s) is selected from promoters such as but not limited to PpilA, PT7, PnirA, Cpcb171 and PcpcB or any combination thereof. Accordingly, in some embodiments, the polynucleotide(s) encoding protein(s) selected from a group comprising exogenous Cyanoglobin, HemA, HemL, HemH and FeoB or any combination thereof are each under the control of the same or different promoter(s) selected from a group comprising PpilA, PT7, PnirA, Cpcb171 and PcpcB or any combination thereof.

The present disclosure also provides a recombinant DNA vector comprising nucleotide(s) encoding any combination of proteins (exogenous cyanoglobin, HemA, HemL, HemH or FeoB) as indicated in Table 1. Said vector may be designed to comprise expression elements operably linked to such nucleotide(s), and further can include sequences such as those encoding a selectable marker. The vector may further comprise other standard features or components of expression vectors examples of which include but are not limited to poly adenylation (polyA) signal, antibiotic resistance gene, origin of replication (ori), restrictions sites and multiple cloning site (MCS). Said recombinant DNA vector can be produced by recombinant DNA techniques routine in the art.

In a non-limiting embodiment, the heme-containing protein is Cyanoglobin, wherein the gene encoding Cyanoglobin is expressed under the control of a T7 promoter.

In another non-limiting embodiment, the genetic modification that increases the heme level in the cell is the expression of HemA from Rhodobacter sphaeroides under the control of PnirA promoter using aadA selection marker.

In another non-limiting embodiment, the genetic modification that increases the heme level in the cell by leading to expression of one or more enzymes in the heme synthesis pathway is overexpression of HemA, HemL or HemH under the control of NirA promoter at Sig-C site and/or under the control of NirA promoter at PsyB site.

In some embodiments, highest expression of HemA, HemL or HemH is observed when expressed under the control of strong promoter Cpcb171.

In some embodiments, highest expression of FeoB is observed when expressed under the control of strong promoter Cpcb171.

In an exemplary embodiment, the genetic modification that increases the heme level in the cell is a combination of expression of HemA from Rhodobacter sphaeroides under the control of PnirA promoter using aadA selection marker and expression of FeoB under the control of strong promoter Cpcb171.

With regard to the above embodiments defining the genetically modified bacteria of the present disclosure, each of the embodiments that refer to ‘Cyanoglobin’ have been provided as a representative of the group of heme containing proteins as defined herein i.e. Cyanoglobin, Hemoglobin and Leghemoglobin. Thus, each of the embodiments directed to the genetically modified bacteria of the present disclosure expressing Cyanoglobin are equally applicable to Hemoglobin and Leghemoglobin and have not been re-iterated for the sake of brevity. A person skilled in the art would be able to understand and appreciate how the embodiments referring to Cyanoglobin are equally applicable to Hemoglobin and Leghemoglobin.

Some exemplary strains of the present disclosure have been submitted with the MTCC. For instance recombinant E. coli strain of the present disclosure, engineered to express cyanoglobin has been assigned accession number MTCC 25477. Recombinant E. coli strain of the present disclosure, engineered to express HemA has been assigned accession number MTCC 25478. Recombinant E. coli strain of the present disclosure, engineered to co-express cyanoglobin and HemA has been assigned accession number MTCC 25479. Recombinant E. coli strain of the present disclosure, engineered to co-express cyanoglobin, HemA and FeoB has been assigned accession number MTCC 25557.

The present disclosure further provides a method to produce the genetically modified bacteria as described above, wherein said method may comprise steps routinely performed in the field of genetic engineering.

In a non-limiting embodiment, provided herein is a method of producing the genetically modified bacteria as described above, comprising—
a) Cloning genes encoding protein(s) selected from a group comprising HemA, HemL, HemH and FeoB or any combination thereof into recombinant DNA vector(s);
b) Transforming the recombinant DNA vector(s) into a bacteria expressing heme-containing protein,
to obtain the genetically modified bacteria.

In some embodiments, the bacteria expressing heme-containing protein expresses an endogenous heme-containing protein or is genetically modified to express an exogenous heme-containing protein.

In some embodiments, when the genetically modified bacteria is bacteria that expresses an endogenous heme-containing protein, the method to produce the genetically modified bacteria of the present disclosure comprises—
a) Cloning genes encoding protein(s) selected from a group comprising HemA, HemL, HemH and FeoB or any combination thereof into recombinant DNA vector(s); and
b) Transforming the recombinant DNA vector(s) into the bacteria expressing endogenous heme-containing protein to obtain the genetically modified bacteria.

In some embodiments, when the genetically modified bacteria is bacteria that expresses an endogenous heme-containing protein, the method to produce the genetically modified bacteria of the present disclosure comprises—
a) Cloning genes encoding FeoB and protein(s) selected from a group comprising HemA, HemL and HemH or any combination thereof into recombinant DNA vector(s); and
b) Transforming the recombinant DNA vector(s) into the bacteria expressing endogenous heme-containing protein to obtain the genetically modified bacteria.

In some embodiments, when the genetically modified bacteria is bacteria that expresses an endogenous heme-containing protein, the method to produce the genetically modified bacteria of the present disclosure comprises—
a) Cloning genes encoding FeoB and HemA into recombinant DNA vector(s); and
b) Transforming the recombinant DNA vector(s) into the bacteria expressing endogenous heme-containing protein to obtain the genetically modified bacteria.

In some embodiments, when the bacteria is bacteria that expresses an exogenous heme-containing protein, the method to produce the genetically modified bacteria of the present disclosure comprises—
a) Cloning genes encoding protein(s) selected from a group comprising the exogenous heme-containing protein, HemA, HemL, HemH and FeoB or any combination thereof into recombinant DNA vector(s); and
b) Transforming the recombinant DNA vector(s) into the bacteria to obtain the genetically modified bacteria.

In some embodiments, when the bacteria is bacteria that expresses an exogenous heme-containing protein, the method to produce the genetically modified bacteria of the present disclosure comprises—
a) Cloning genes encoding the exogenous heme-containing protein, FeoB and protein(s) selected from a group comprising HemA, HemL and HemH or any combination thereof into recombinant DNA vector(s); and
b) Transforming the recombinant DNA vector(s) into the bacteria to obtain the genetically modified bacteria.

In some embodiments, when the bacteria is bacteria that expresses an exogenous heme-containing protein, the method to produce the genetically modified bacteria of the present disclosure comprises—
a) Cloning genes encoding the exogenous heme-containing protein, FeoB and HemA, into recombinant DNA vector(s); and
b) Transforming the recombinant DNA vector(s) into the bacteria to obtain the genetically modified bacteria.

In some embodiments, when the bacteria is bacteria that expresses a heterologous heme-containing protein, the method to produce the genetically modified bacteria of the present disclosure comprises—
a) Cloning genes encoding protein(s) selected from a group comprising heterologous heme-containing protein, HemA, HemL, HemH and FeoB or any combination thereof into recombinant DNA vector(s); and
b) Transforming the recombinant DNA vector(s) into the bacteria to obtain the genetically modified bacteria.

In some embodiments, the bacteria is bacteria that expresses an endogenous Cyanoglobin and thus the method to produce the genetically modified bacteria of the present disclosure comprises—
a) Cloning genes encoding protein(s) selected from a group comprising HemA, HemL, HemH and FeoB or any combination thereof into recombinant DNA vector(s); and
b) Transforming the recombinant DNA vector(s) into the bacteria expressing endogenous Cyanoglobin to obtain the genetically modified bacteria.

In some embodiments, the bacteria is bacteria that expresses an endogenous Cyanoglobin and thus the method to produce the genetically modified bacteria of the present disclosure comprises—
a) Cloning genes encoding FeoB and protein(s) selected from a group comprising HemA, HemL and HemH or any combination thereof into recombinant DNA vector(s); and
b) Transforming the recombinant DNA vector(s) into the bacteria expressing endogenous Cyanoglobin to obtain the genetically modified bacteria.

In some embodiments, the bacteria is bacteria that expresses an endogenous Cyanoglobin and thus the method to produce the genetically modified bacteria of the present disclosure comprises—
a) Cloning genes encoding FeoB and HemA or any combination thereof into recombinant DNA vector(s); and
b) Transforming the recombinant DNA vector(s) into the bacteria expressing endogenous Cyanoglobin to obtain the genetically modified bacteria.

In some embodiments, the method to produce the genetically modified bacteria of the present disclosure comprises—
a) Cloning genes encoding protein(s) selected from a group comprising exogenous Cyanoglobin, HemA, HemL, HemH and FeoB or any combination thereof into recombinant DNA vector(s); and
b) Transforming the recombinant DNA vector(s) into the bacteria to obtain the genetically modified bacteria;
   wherein the bacteria is Cyanobacteria or E. coli.

In some embodiments, the method to produce the genetically modified bacteria of the present disclosure comprises—
a) Cloning genes encoding exogenous Cyanoglobin, FeoB and protein(s) selected from a group comprising HemA, HemL and HemH or any combination thereof into recombinant DNA vector(s); and
b) Transforming the recombinant DNA vector(s) into the bacteria to obtain the genetically modified bacteria;
   wherein the bacteria is Cyanobacteria or E. coli.

In some embodiments, the method to produce the genetically modified bacteria of the present disclosure comprises—
a) Cloning genes encoding exogenous Cyanoglobin, FeoB and HemA into recombinant DNA vector(s); and
b) Transforming the recombinant DNA vector(s) into the bacteria to obtain the genetically modified bacteria;
   wherein the bacteria is Cyanobacteria or E. coli.

In some embodiments, the method to produce the genetically modified bacteria of the present disclosure comprises—
a) Cloning genes encoding protein(s) selected from a group comprising heterologous Cyanoglobin, HemA, HemL, HemH and FeoB or any combination thereof into recombinant DNA vector(s); and
b) Transforming the recombinant DNA vector(s) into the bacteria to obtain the genetically modified bacteria
   wherein the bacteria is Cyanobacteria or E. coli.

In some embodiments, when the genetically modified bacteria is Cyanobacteria, the bacteria expressing heme-containing protein is engineered to knockout Cyanophycin synthetase (CphA) and Cyanophycinase (CphB).

Accordingly, in some embodiments, when the genetically modified bacteria is Cyanobacteria, the method to produce the genetically modified Cyanobacteria of the present disclosure comprises
a) Cloning genes encoding protein(s) selected from a group comprising exogenous Cyanoglobin, HemA, HemL, HemH and FeoB or any combination thereof into recombinant DNA vector(s);
b) Transforming the recombinant DNA vector(s) into the Cyanobacteria; and
c) Knocking out Cyanophycin synthetase (CphA) and Cyanophycinase (CphB) proteins in the Cyanobacteria
   to obtain the genetically modified Cyanobacteria.

In some embodiments, when the genetically modified bacteria is Cyanobacteria, the method to produce the genetically modified Cyanobacteria of the present disclosure comprises—
a) Cloning genes encoding exogenous Cyanoglobin, FeoB and protein(s) selected from a group comprising HemA, HemL and HemH or any combination thereof into recombinant DNA vector(s);
a) Transforming the recombinant DNA vector(s) into the Cyanobacteria; and
b) Knocking out Cyanophycin synthetase (CphA) and Cyanophycinase (CphB) proteins in the Cyanobacteria
   to obtain the genetically modified Cyanobacteria.

In some embodiments, when the genetically modified bacteria is Cyanobacteria, the method to produce the genetically modified Cyanobacteria of the present disclosure comprises—
a) Cloning genes encoding exogenous Cyanoglobin, FeoB and HemA into recombinant DNA vector(s);
b) Transforming the recombinant DNA vector(s) into the Cyanobacteria; and
c) Knocking out Cyanophycin synthetase (CphA) and Cyanophycinase (CphB) proteins in the Cyanobacteria
   to obtain the genetically modified Cyanobacteria.

Accordingly, any step or sequence of steps followed, such as but not limited to creation of a vector, or obtaining a commercially available vector, or insertion of the respective sequences in a prepared or commercially obtained vector, or employing multiple separate vectors prepared or commercially obtained or any combination thereof as mentioned above, are all within the purview of the present disclosure and will be readily understood and followed by a person skilled in the art. Further, techniques for transformation are well known and described in the technical and scientific literature.

Thus, variations or changes in the method steps are mere alternatives to arrive at the genetically modified bacteria of the present disclosure and the steps recited above by no means act as limitations of any kind.

In some embodiments, starting with a basic vector backbone containing an initial set of elements such as but not limited to ori, promoter sequence, multiple cloning site and resistance marker, additional components that are to be inserted into the basic vector to form the platform are inserted into the backbone through any method routinely practiced in the art.

Once obtained, the vector is transformed into the bacteria by any technique well known in the art, to obtain the genetically modified bacteria. Successful transformation may be confirmed by basing reliance on selectable markers present in the vector.

In some embodiments, when the genetically modified microorganism is Cyanobacteria, and when no additional Cyanoglobin gene is inserted and the expression of Cyanoglobin stems from an endogenous gene, the aforesaid method may further comprise engineering the Cyanobacteria to trigger overexpression of the endogenous Cyanoglobin protein. Said trigger may be one or more of intrinsic or extrinsic trigger(s).

Accordingly, in some embodiments, the method to produce the genetically modified Cyanobacteria of the present disclosure comprises—
a) Cloning genes encoding protein(s) selected from a group comprising HemA, HemL, HemH and FeoB or any combination thereof into recombinant DNA vector(s);
b) Transforming the recombinant DNA vector(s) into the Cyanobacteria; and
c) Triggering overexpression of endogenous Cyanoglobin to obtain the genetically modified Cyanobacteria.

In some embodiments, the method to produce the genetically modified Cyanobacteria of the present disclosure comprises—
a) Cloning genes encoding protein(s) selected from a group comprising HemA, HemL, HemH and FeoB or any combination thereof into recombinant DNA vector(s);
b) Transforming the recombinant DNA vector(s) into the Cyanobacteria;
c) Triggering overexpression of endogenous Cyanoglobin; and
d) Knocking out Cyanophycin synthetase (CphA) and Cyanophycinase (CphB) proteins in the Cyanobacteria to obtain the genetically modified Cyanobacteria.

In some embodiments, the steps of the aforesaid method(s) may be performed in any order.

With regard to the above embodiments defining the method of obtaining the genetically modified bacteria of the present disclosure, each of the embodiments that refer to ‘Cyanoglobin’ have been provided as a representative of the group of heme containing proteins as defined herein i.e. Cyanoglobin, Hemoglobin and Leghemoglobin. Thus, each of the embodiments directed to a method of obtaining the genetically modified bacteria expressing ‘Cyanoglobin’ are equally applicable to Hemoglobin and Leghemoglobin and have not been re-iterated for the sake of brevity. A person skilled in the art would be able to understand and appreciate how the embodiments referring to Cyanoglobin are equally applicable to Hemoglobin and Leghemoglobin.

As will be readily understood from this disclosure, one of the key objectives of the present disclosure is to provide a genetically modified bacteria that may be employed for the production of the heme-containing protein.

Accordingly, further provided herein is a method for producing a heme-containing protein such as but not limited to Cyanoglobin, said method comprising culturing the recombinant bacteria described above in a culture medium under conditions that allow said heme-containing polypeptide to be secreted into said culture medium.

In an embodiment, the Cyanoglobin is synthesized by growing cultures of the genetically modified bacterial cells.

Therefore, provided herein is a method of producing heme-containing protein in a genetically modified bacteria, comprising:
a) growing the genetically modified bacteria described above under conditions conducive for expression of the heme-containing protein; and
b) obtaining the heme-containing protein from the genetically modified bacteria.

In some embodiments, when the heme-containing protein is an endogenous heme-containing protein, the genetically modified bacteria is triggered to overexpress the endogenous heme-containing protein.

In an embodiment, the method of producing heme-containing protein in a genetically modified bacteria, comprises:
a) growing the genetically modified bacteria described above under conditions conducive for expression of the heme-containing protein and optionally, enhancement of heme level in the genetically modified bacteria; and
b) obtaining the heme-containing protein from the genetically modified bacteria.

In some embodiments, the method of producing heme-containing protein in a genetically modified bacteria, comprises:
a) growing the genetically modified bacteria described above under conditions conducive for expression of the heme-containing protein; and
b) obtaining the heme-containing protein from the genetically modified bacteria; wherein the heme-containing protein is an endogenous heme-containing protein, and the genetically modified bacteria is triggered to overexpress the endogenous heme-containing protein.

In some embodiments, the produced heme-containing protein is subjected to purification.

In some embodiments, the method of producing heme-containing protein in a genetically modified bacteria, comprises:
a) growing the genetically modified bacteria described above under conditions conducive for expression of the heme-containing protein;
b) obtaining the heme-containing protein from the genetically modified bacteria; and
c) subjecting the obtained heme-containing protein to purification.

Methods of growing said cells are well known to persons skilled in the art. In some embodiments, said methods are performed in conditions conductive (non-competitive) for growth. In some embodiments, in addition to temperature, parameters such as but not limited to aeration, redox potential and oxygen availability are regulated to facilitate expression or overexpression of the heme-containing protein.

In an embodiment, culturing of the genetically modified bacteria is carried out in presence of aeration.

In an embodiment, culturing of the genetically modified bacteria is carried out at a temperature from about 15° C. to 40° C.

In still another embodiment, culturing of the genetically modified recombinant bacteria is carried out for a time-period ranging from about 5 hours to 10 days.

In still another embodiment, culturing of the genetically modified the genetically modified bacteria is carried out at a relative humidity of about 60% to 80%.

In still another embodiment of the present method, culturing of the genetically modified bacteria is carried out by employing a CO₂/air mixture of about 1% to 4% (v/v) CO₂ in air.

In still another embodiment of the present method, culturing the genetically modified bacteria is carried out pH of about 6 to about 8.

In still another embodiment of the present method, said method comprises culturing the genetically modified bacteria as described herein under batch mode, fed-batch mode, semi-turbidostatic mode, or any combinations thereof.

In an exemplary embodiment, the present method comprises culturing the genetically modified bacteria under batch mode of cultivation.

In another exemplary embodiment, the present method comprises culturing the genetically modified recombinant Cyanobacteria described herein under semi-turbidostatic mode of cultivation.

In yet another exemplary embodiment, the present method comprises culturing the genetically modified bacteria as described herein under fed-batch mode of cultivation.

In some embodiments, in the method of producing heme-containing protein from the genetically modified bacteria, the step of culturing the genetically modified bacteria employs culture medium such as but not limited to Terrific broth (TB) medium and LB Broth or a combination thereof.

In some embodiments, pH of the culture medium ranges from about 6 to about 8.

In an exemplary embodiment, the Terrific broth (TB) medium comprises about 11.8 g Peptone, about 23.6 g Yeast Extract, about 9.4 g dipotassium hydrogen phosphate and about 2.2 g potassium dihydrogen phosphate at pH about 7.2±0.2 at about 25° C.

In some embodiments, the cultures are grown indoors or outdoors. In some embodiments, the cultures are grown outdoors in an open pond type of photobioreactor or in closed photobioreactor.

The ability to grow the cultures in outdoor ponds allows utilization of even infertile land for protein production and further provides the advantage of avoiding competition to edible crops on fertile land.

In some embodiments, the light cycle for culture can be set as continuous light, or periodic exposure to light.

In some embodiments, the expressed heme-containing protein is obtained in soluble form without going into inclusion bodies which is critical for down-stream processing as well as functionality. In some embodiments, this allows for easy up-scaling of the production of heme-containing protein such as Cyanoglobin.

In some embodiments, once produced, the heme-containing protein is subjected to purification by methods such as but not limited to chromatography, heating, DCM method, PEG method and dual aqueous phase extraction.

Said purification methods are routinely performed in the art. In some embodiments, said purification methods are tailored to the requirements of the present disclosure to provide optimum results.

In another embodiment, when the genetically modified recombinant microorganism is genetically modified recombinant Cyanobacteria, said genetically modified recombinant Cyanobacteria achieves enhanced Cyanoglobin production relative to corresponding wild-type/parent Cyanobacteria.

In some embodiments, the genetically modified bacteria are obtained as monocultures when cultivated in fermenters or photobioreactors. In case of open pond cultures, in some non-limiting embodiments, there may be contamination up to degree of about 10%.

In some embodiments, the genetically modified bacteria of the present disclosure is provided with the objective of facilitating production of heme-containing protein such as but not limited to Cyanoglobin, said production preferably being scalable to an industrial scale.

In some embodiments, the genetically modified recombinant microorganism of the present disclosure provides an increase of at least about 20% in the yield of heme-containing protein such as but not limited to Cyanoglobin, as compared to a wild-type strain.

In some embodiments, when the genetically modified recombinant microorganism is engineered to express the heme-containing protein alone, it provides an increase of at least about 20% in the yield of heme-containing protein such as but not limited to Cyanoglobin, as compared to a wild-type strain.

In a non-limiting embodiment, when the genetically modified recombinant microorganism is engineered to express the heme-containing protein alone, it provides an increase of about 20% to about 30% in the yield of heme-containing protein such as but not limited to Cyanoglobin, as compared to a wild-type strain.

In a non-limiting embodiment, when the genetically modified recombinant microorganism is engineered to express the heme-containing protein alone, it provides an increase of about 20%, about 21%, about 22%, about 23%, about 24%, about 25%, about 26%, about 27%, about 28%, about 29% or about 30% in the yield of heme-containing protein such as but not limited to Cyanoglobin, as compared to a wild-type strain.

In some embodiments, when the genetically modified recombinant microorganism is engineered to co-express the heme-containing protein along with one or more of HemA, HemL and HemH, it provides an increase of at least about 25% in the yield of heme-containing protein such as but not limited to Cyanoglobin, as compared to a wild-type strain.

In a non-limiting embodiment, when the genetically modified recombinant microorganism is engineered to co-express the heme-containing protein along with one or more of HemA, HemL and HemH, it provides an increase of about 25% to about 35% in the yield of heme-containing protein such as but not limited to Cyanoglobin, as compared to a wild-type strain.

In a non-limiting embodiment, the genetically modified recombinant microorganism provides an increase of about 25%, about 26%, about 27%, about 28%, about 29%, about 30%, about 31%, about 32%, about 33%, about 34% or about 35% in the yield of heme-containing protein such as but not limited to Cyanoglobin, as compared to a wild-type strain.

In some embodiments, when the genetically modified recombinant microorganism is engineered to co-express the heme-containing protein along with one or more of HemA, HemL and HemH, it provides an increase of at least about 5% in the yield of heme-containing protein such as but not limited to Cyanoglobin, as compared to the genetically modified recombinant microorganism is engineered to express the heme-containing protein alone.

In some embodiments, when the genetically modified recombinant microorganism is engineered to co-express the heme-containing protein and FeoB along with one or more of HemA, HemL and HemH, it provides an increase of at least about 30% in the yield of heme-containing protein such as but not limited to Cyanoglobin, as compared to a wild-type strain.

In a non-limiting embodiment, when the genetically modified recombinant microorganism is engineered to co-express the heme-containing protein and FeoB along with one or more of HemA, HemL and HemH, it provides an increase of about 30% to about 50% in the yield of heme-containing protein such as but not limited to Cyanoglobin, as compared to a wild-type strain.

In a non-limiting embodiment, when the genetically modified recombinant microorganism is engineered to co-express the heme-containing protein and FeoB along with one or more of HemA, HemL and HemH, it provides an increase of about 30%, about 32%, about 34%, about 36%, about 38%, about 40%, about 42%, about 44%, about 46%, about 48% or about 50% in the yield of heme-containing protein such as but not limited to Cyanoglobin, as compared to a wild-type strain.

In some embodiments, when the genetically modified recombinant microorganism is engineered to co-express the heme-containing protein and FeoB along with one or more of HemA, HemL and HemH, it provides an increase of at least about 10% in the yield of heme-containing protein such as but not limited to Cyanoglobin, as compared to the genetically modified recombinant microorganism is engineered to express the heme-containing protein alone.

In some embodiments, when the genetically modified recombinant microorganism is engineered to co-express the heme-containing protein and FeoB along with one or more of HemA, HemL and HemH, it provides an increase of at least about 5% in the yield of heme-containing protein such as but not limited to Cyanoglobin, as compared to the genetically modified recombinant microorganism is engineered to co-express the heme-containing protein along with one or more of HemA, HemL and HemH.

With regard to the above embodiments defining the method of producing heme-containing protein employing the genetically modified bacteria of the present disclosure, each of the embodiments that refer to ‘Cyanoglobin’ have been provided as a representative of the group of heme containing proteins as defined herein i.e. Cyanoglobin, Hemoglobin and Leghemoglobin. Thus, each of the embodiments directed to the method of producing ‘Cyanoglobin’ employing the genetically modified bacteria of the present disclosure are equally applicable to Hemoglobin and Leghemoglobin and have not been re-iterated for the sake of brevity. A person skilled in the art would be able to understand and appreciate how the embodiments referring to the method of producing Cyanoglobin are equally applicable to similar methods for producing Hemoglobin and Leghemoglobin.

Leveraging the genetically modified bacteria as elaborately described above, the present disclosure further provides a heme-containing protein produced by the genetically modified bacteria.

The present disclosure further relates to a product comprising the heme-containing protein described above.

In some embodiments, provided herein is a product comprising the genetically modified bacteria as described above.

Said product, in some embodiments is a food product. In some embodiments, the food product is a nutritional food product. In some embodiments, the food product comprises the genetically modified bacteria in the form of a fortificant or additive to increase the overall iron and protein content of the food product.

Accordingly, in some embodiments, provided herein is a nutritional food product comprising the genetically modified bacteria as described above or the heme-containing protein produced therefrom.

In some embodiments, further envisaged herein is use of the genetically modified bacteria as described above or the heme-containing protein produced therefrom to prepare a food product. In some embodiments, the food product is a nutritional food product.

In some embodiments, the product is a meat substitute. Said product, in some embodiments, has a red or reddish colour.

In some embodiments, said product additionally comprises substances such as but not limited to flavour enhancing agents, flavour masking agents, stabilizers, anti-oxidants, edible ingredients, texturizing agents, food colouring agents, nutrient additives, fortifiers, and preservatives.

Accordingly, envisaged herein is a meat substitute comprising or consisting of the genetically modified bacteria of the present disclosure or the heme-containing protein produced therefrom.

In some embodiments, provided herein is a pharmaceutical product comprising the genetically modified bacteria as described above or the heme-containing protein produced therefrom. Without intending to be limited by theory, in some embodiments, such pharmaceutical products have the potential to increase blood hemoglobin levels, for cure of anemia.

In some embodiments, the food or pharmaceutical products as described above contain the genetically modified bacteria in the form of dried biomass. Accordingly, further envisaged in the present disclosure is a dried biomass containing the genetically modified bacteria as defined above.

Said dried biomass, in some embodiments, may be formulated into a dried meal or bulk protein.

Accordingly provided herein is a dried meal (or bulk protein) obtained from the genetically modified bacteria of the present disclosure.

Also envisaged herein is a partially purified heme-containing protein, obtained from the genetically modified bacteria of the present disclosure. In some embodiments, purity of the partially purified heme-containing protein ranges from about 50% to about 85%.

The present disclosure further provides an iron-rich nutritional supplement comprising a purified heme-containing protein obtained from the genetically modified bacteria of the present disclosure, or a partially purified heme-containing protein obtained from the genetically modified bacteria of the present disclosure, or dried biomass containing the genetically modified bacteria of the present disclosure, or a dried meal (or bulk protein) obtained from the genetically modified bacteria of the present disclosure.

Additional embodiments and features of the present disclosure will be apparent to one of ordinary skill in art based upon description provided herein. The embodiments herein provide various features and advantageous details thereof in the description. Descriptions of well-known/conventional methods and techniques are omitted to not unnecessarily obscure the embodiments herein. Further, the disclosure herein provides for examples illustrating the above described embodiments, and in order to illustrate the embodiments of the present disclosure certain aspects have been employed. The examples used herein are intended merely to facilitate an understanding of ways in which the embodiments herein may be practiced and to further enable those of skill in the art to practice the embodiments herein.

Although the disclosure will be described with reference to specific examples it will be appreciated by those skilled in the art that the disclosure may be embodied in many other forms.

Accordingly, the following examples should not be construed as limiting the scope of the embodiments herein.

### EXAMPLES

### Materials and Methods

In the present disclosure/examples, the Cyanobacterium employed is Cyanobacterium aponinum. The Cyanobacterium aponinum employed in present examples was originally isolated/sourced from Gagva, Gujarat, India. Said strain is referred to as the wild-type or parent Cyanobacterium aponinum strain, while the corresponding genetically modified strain is referred to as the recombinant or mutant strain.

The wild-type E. coli employed in the present examples was originally purchased from NEB.

The strain(s) of the present disclosure is being deposited with an appropriate depository. Details of deposition shall be furnished appropriately.

While reference to most routinely performed methods follow standard protocols unless otherwise defined, provided below are brief descriptions of some specific steps followed in the examples that follow.

Dialysis—Cell free extract was kept in a dialysis bag in a beaker filled with water. The water of the beaker was kept under continuous stirring at about 100 rpm in the cold room. The MilliQ (MQ) water in the beaker was replaced by fresh MQ every day for 3 days.

Concentration—Cell free extract was filtered through 3 KD membrane filter for exchange of buffer, dialysis or concentration of protein in desired buffer or MQ.

Lyophilization—The dialyzed Cyanoglobin was taken in about 50 mL centrifuge tubes and rotated lengthwise on liquid nitrogen in an aluminum tray. Once the sample was completely frozen, the tubes were kept in the lyophilizer till the sample was completely dry.

ICP Analysis for iron estimation—Purified Cyanoglobin (300-400 mg of liquid sample) was mixed with about 1.5 mL Nitric acid, about 0.5 mL HCl and about 0.5 mL hydrogen peroxide. The mixture was microwave digested at about 180° C. for about 1 hour. The volume of the sample was made up to about 10 mL and injected in to ICP-OES instrument. Iron levels in the sample were determined in ppm.

### Example 1: Construction of E. coli Strain Overexpressing Both Cyanoglobin and HemA Using Recombinant Plasmid pPS16 (pET28a-Cyanoglobin-hemARs)

The hem_{ARs} gene codon optimized for E. coli (SEQ ID No. 5) was synthesized and used as a template for amplifying it such that the resulting amplicon had XhoI restriction sites at both the ends.

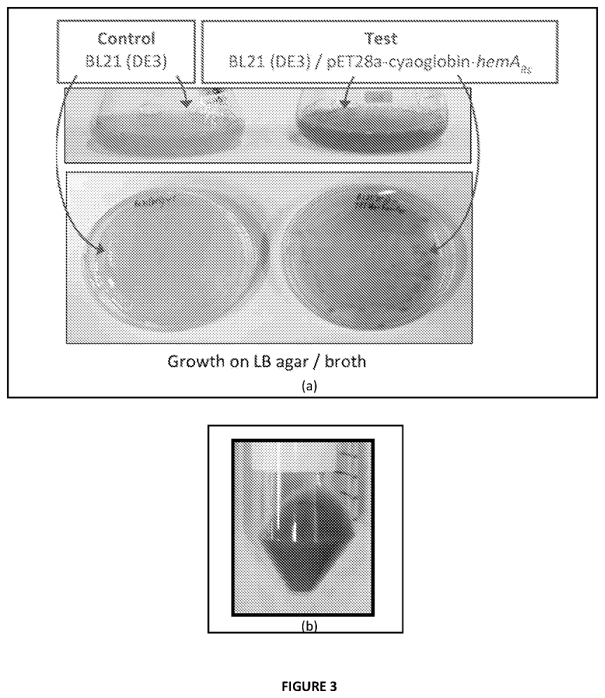

In parallel, E. coli NEBR 5-alpha cells (procured from NEB) harbouring the plasmid construct pET28a-Cyanoglobin were cultivated and subjected to plasmid extraction using the plasmid miniprep kit (Qiagen, 27104). The obtained plasmid and the amplified hemARs gene were independently digested by using restriction enzyme XhoI (NEB, R0146 S) and purified using the gel extraction kit (Qiagen, 28704). Both the linear DNA fragments were ligated using T4 DNA ligase (NEB, M0202 S) to result in plasmid pPS16 (SEQ ID No. 21) (FIG. 2) and transformed in competent E. coli NEB® 5-alpha cells (NEB, C2987I) to obtain several transformants on LB agar plates containing about 50 μg/mL kanamycin. The obtained transformants were screened for true positives by using colony PCR and the confirmed recombinant strain PS55 (E. coli NEB® 5-alpha/pPS16) was preserved at about −80° C. as glycerol stock. The recombinant plasmid was isolated from PS55 strain using the plasmid miniprep kit (Qiagen, 27104) and transformed in competent BL21 (DE3) strain (NEB, C2527I) to obtain recombinant E. coli strain (E. coli BL21 (DE3)/pPS16-MTCC 25479) on LB agar plates containing about 50 μg/mL kanamycin. The recombinant E. coli strain exhibited distinct red coloured colonies when grown on LB agar plate (HiMedia, M1151) and also imparted red coloration during growth in LB broth (FIG. 3).

### Example 2: Construction of Cyanobacterial Strain for HemA Overexpression Using Integrative Plasmid pPS23

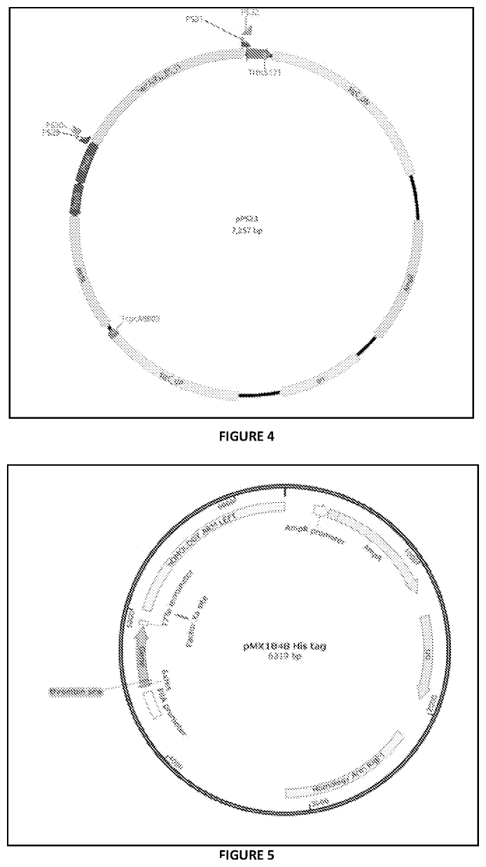

hem_{ARs} gene codon optimized for the Cyanobacteria (SEQ ID No. 7) was synthesized (Invitrogen) and subsequently amplified along with overlapping ends compatible with prospective adjoining sequences. Similarly, regions upstream and downstream of the sigC locus, aadA selection marker and P_{mirA} promoter were also amplified individually. All the amplified fragments were mixed as per the Gibson Assembly® cloning kit (NEB, E5510 S) protocol to result in E. coli transformants harboring the plasmid pPS23 (SEQ ID No. 22) (FIG. 4) on LB agar plates containing 100 μg/mL ampicillin. True positives were confirmed by using colony PCR followed by restriction digestion of the plasmid extracted using the plasmid miniprep kit (Qiagen, 27104). The recombinant E. coli strain constructed PS57 (E. coli NEB® 5-alpha/pPS23) was preserved at −80° C. as glycerol stock. The extracted plasmid pPS23 was further used for transformation of Cyanobacterium aponinum strain (strain collected from Gagva, Jamnagar, Gujarat) as per the standardized protocol (FIG. 5). The recombinants obtained on BG11 plates containing 1.5 μg/mL each of spectinomycin and streptomycin were screened by colony PCR. The partially-segregated but confirmed recombinant Cyanobacterium aponinum strain was preserved at −80° C.

### Example 3: Induction of Cyanoglobin Expression in E. coli

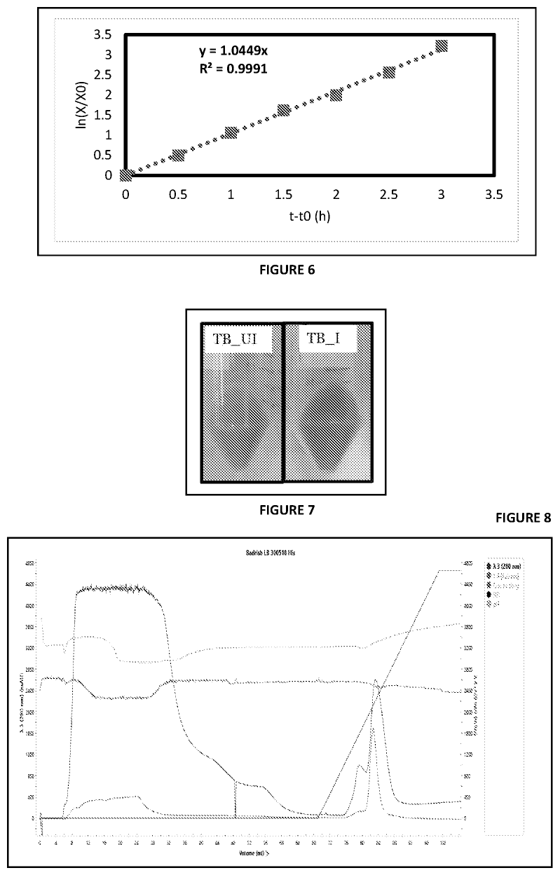

About 200 ml of Terrific broth (TB) medium was inoculated in a 1 litre flask with pre-inoculum of recombinant E. coli strain as obtained in Example 1 (overnight grown in TB medium from glycerol stock). The starting OD of this culture at 2600 nm was about 0.05. The culture was grown at about 37° C. at about 200 rpm till the OD at 2600 nm was about 2.5 to 3.0. Once the OD reached about 2.5 to 3.0, the culture was induced with about 1 mM IPTG and incubated at about 20° C. overnight. The growth curve for growth of cells was observed in TB media is depicted in FIG. 6.

After induction, the culture was pelleted down at about 5000 rpm for about 10 minutes. The supernatant was decanted, and the cells were washed once with MQ water. The induced pellet was brick red in colour as depicted in FIG. 7.

The cells were lysed using homogeniser/B-PER/sonication, wherein protease inhibitor and benzonase nuclease were added in the B-PER/buffer used for resuspending the pellet for homogenisation/sonication. The lysed cells were centrifuged at about 15000×g for about 15 minutes to obtain cell free lysate. OD was measured at 420 nm against water control. The cell free lysate was then subjected to affinity purification.

### Example 4: Purification of Cyanoglobin Expressed in E. coli

### A. Affinity Purification:

About 20 grams of wet pellet was obtained from about IL culture of Example 3, wherein Cyanoglobin protein was expressed in an amount of about 5-30% of total soluble proteins. After harvesting and centrifugation, the pellet was dissolved in Binding buffer (100 ml) with protease inhibitor & Benzonase. Cells were lysed at about 30,000 PSI by Stansted Homogenizer and passed twice for complete lysis assurance. After lysis, the lysate was spun at about 9000 RPM for about 30 minutes at about 4° C. After Centrifugation, the red brick colored supernatant was collected.

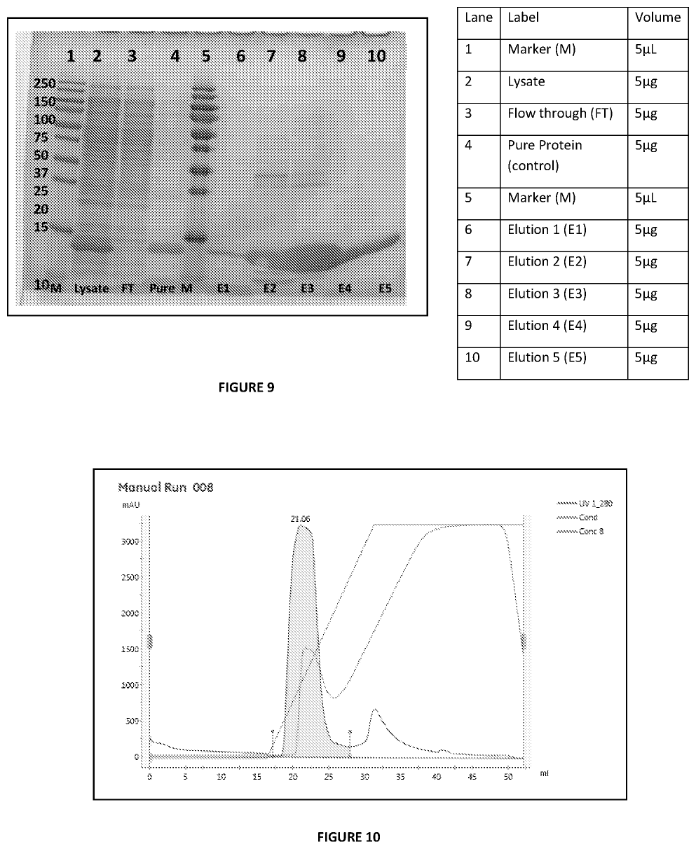

The protein of interest (Cyanoglobin) was tagged with histidine at C terminal. Cyanoglobin was then captured with Nickel Sepharose 6 fast flow. Binding buffer used for the process was about 20 mM phosphate buffer+about 40 mM imidazole+about 0.5 M Nacl & the elution buffer used was about 20 mM phosphate buffer+about 500 mM imidazole+about 0.5 M NaCl. An elution gradient from 0 to 100% was provided. Fractions were collected according to the peaks. Fractions were pooled as per the SDS PAGE based on the purity analysis. As can be seen from FIGS. 8 and 9, about 95% pure protein was obtained with about 98% process efficiency in terms of recovery or loss of the protein of interest i.e. Cyanoglobin. The final pooled solution was dialyzed in Distilled water/phosphate buffer in order to reduce salts from mixture. Dialyzed solution was subjected to lyophilization to obtain final powder form of protein.

### B. Ion Exchange Chromatography Based Purification:

About 20 grams of wet pellet was obtained from about 1 L culture wherein about 5-30% of total soluble proteins expressed was Cyanoglobin. After harvesting and centrifugation, the pellet was dissolved in about 100 ml binding buffer with protease inhibitor & benzonase. Cells were lysed at about 30,000 PSI by Stansted Homogenizer and passed twice for complete lysis assurance. After lysis the lysate was spun at about 9000 RPM for about 30 minutes at about 4° C. After centrifugation, red brick colored supernatant was collected.

Purification: Cyanoglobin was captured by charge effect. Binding buffer used for the process was about 20 mM phosphate buffer & elution buffer was about 20 mM phosphate buffer+about 1 M Nacl. An elution gradient from about 0% to about 100% was provided. Fractions were collected according to the peaks. Hence, about 50-80% pure protein was achieved with about 88% process efficiency (FIG. 10). Final pooled solution was dialyzed in Distilled water/phosphate buffer in order to reduce salts from mixture. The dialyzed solution was subjected to lyophilization to obtain final powder form of protein.

### C. Purification by Non-Chromatographic Method:

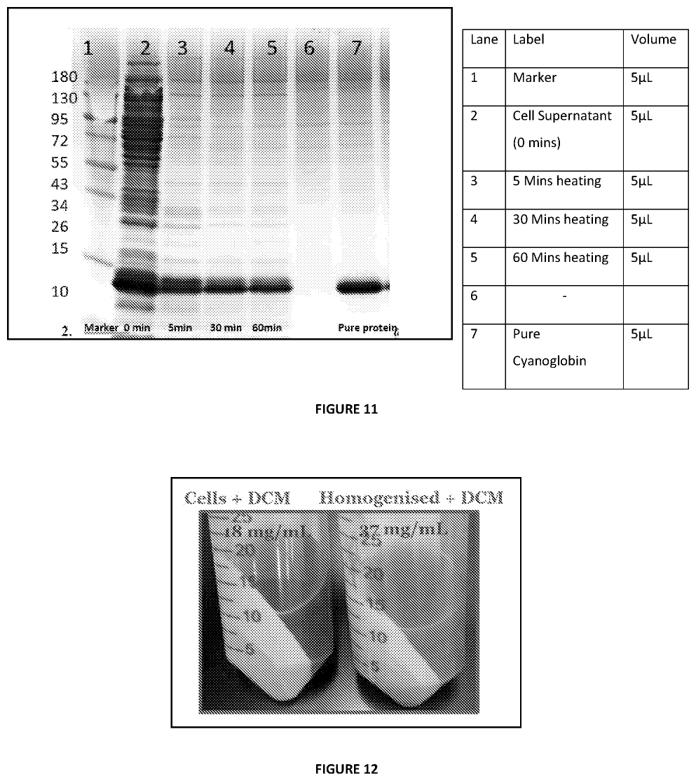

After harvesting, the cell pellet was dissolved in about 20 mM phosphate buffer. Dissolved Pellets were homogenized in Stansted homogenizer at about 30,000 PSI for two rounds. The entire soup was centrifuged at about 9000 RPM, for about 60 minutes at about 4° C. After centrifugation, cell pellet was discarded and reddish supernatant was collected. Said supernatant was heated at about 80° C. for about 30 minutes. The supernatant got turbid and clumps were formed. Except Cyanoglobin, all others protein were precipitated. The precipitated protein was removed by centrifugation at about 9000 RPM, for about 30 minutes at about 4° C. In this way, purity of the protein of interest i.e. Cyanoglobin was improved (FIG. 11).

### D. Purification by DCM Method:

Cells from 250 mL culture were suspended in about 10 mL of phosphate buffer at pH of about 7.4. About 2 mL of dichloromethane (DCM) was added and mixed by manual shaking for about 3 minutes. The sample was centrifuged for about 20 minutes at about 1900×g. At the end of the spin, three layers were formed, bottom, DCM layer; middle, membrane and lipid layer and top, Cyanoglobin layer in phosphate buffer. The top layer was pipetted out and analyzed for protein purity and Fe concentration. The method was used on homogenized/lysed and unhomogenized/unlysed E. coli cells. FIG. 12 depicts the result on lysed and unlysed cells. The protein concentration in the lysed (homogenized) and unlysed sample was about 37 mg/mL and about 18 mg/mL, respectively.

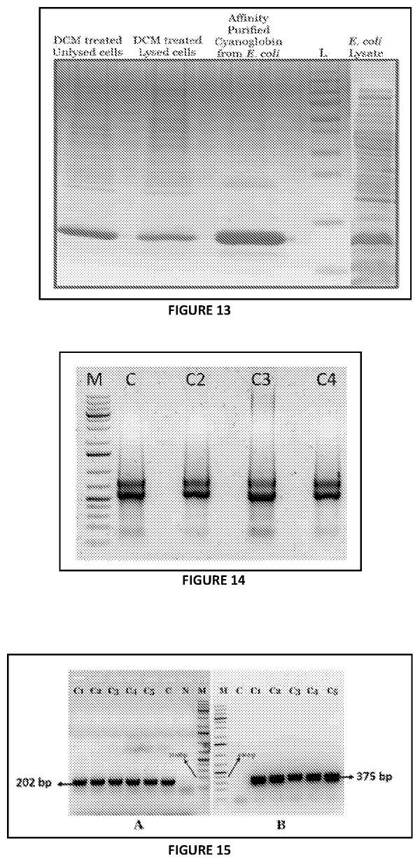

The extracted sample in the top layer was run on PAGE gel (16%) to check the purity of the sample (FIG. 13). The lysed/homogenised sample had many contaminating proteins resulting in a weak Cyanoglobin band. However, the Cyanoglobin band was enriched in the unhomogenized cells sample. Thus, this method did not require the energy intensive homogenization process. Using ICP-OES analysis, Cyanoglobin extract from DCM treated unlysed cells was found to contain about 5.7 ppm of iron.

### E. Purification by Two-Step Dual Aqueous Phase Extraction:

The E. coli cells were homogenized in phosphate buffer (with benzonase nuclease and protease inhibitor). The lysate was centrifuged to remove cell debris. The cell free lysate at pH 10 was added with about 18.9% of Polyethylene glycol (MW-1500). The mixture was stirred for about 5-10 minutes and the phases were allowed to separate for about 30 minutes. The upper PEG phase was removed and mixed with phosphate buffer at about pH 7. The concentration of PEG was adjusted to about 15.5%. The mixture was stirred for about 5 to about 10 minutes and the phases were allowed to separate for about 20-30 minutes. The two layers were separated. The lower phosphate buffer layer at about pH 7 contained Cyanoglobin.

### Example 5: Purification of Cyanoglobin Expressed in Cyanobacteria

### A. Purification by Chromatography:

C. aponinum was inoculated at 0.2 OD in 1 litre UPA media with 45 ppm of urea and 14 ppm of nitrate. The culture was harvested at final OD of 4. Pellet was stored at −80° C. till homogenization. Protease Inhibitor and Benzonase Nuclease were added while resuspending the pellet in binding buffer. The cells were homogenized using single shot at 30 Kpsi under chilling conditions. The supernatant was loaded on to the gravity affinity chromatography column (1 mL column volume). The elute fraction obtained after affinity purification was a very yellowish pink coloured elute fraction. The iron content in this fraction was found to be 2.3 ppm. The fractions were collected and run on a 16% PAGE gel at 100-125 V.

For the confirmation of the Cyanoglobin protein in the elute, a western blotting was performed using HIS tagged antibody for probing. Clear bands were observed at the level of 14 KD in the elute fraction and positive control.

Since, the expression of Cyanoglobin was very low in C. aponinum, the Cyanoglobin band could not be detected in the lysate fraction on western blot. The elute showed very clear bands on western blot. Using ICP-OES analysis, it was determined that the affinity purified Cyanoglobin from C. aponinum had approximately 2.3 ppm of iron attached.

### B. Purification by Two-Step Dual Aqueous Phase Extraction:

The Cyanoglobin cells were homogenized in phosphate buffer (with benzonase nuclease and protease inhibitor). The lysate was centrifuged to remove cell debris. The cell free lysate at pH 10 was added with 18.9% of Polyethylene glycol (MW-1500). The mixture was stirred for about 5-10 mins and the phases were allowed to separate for about 30 mins. The upper PEG phase was removed and mixed with phosphate buffer at about pH 7. The concentration of PEG is adjusted to about 15.5%. The mixture is stirred for about 5 to about 10 mins and the phases were allowed to separate for about 20-30 mins. The two layers were separated. The lower phosphate buffer layer at pH 7 contained Cyanoglobin.

### Example 6: Expression Analysis of E. coli Strains Producing Cyanoglobin

The E. coli strains expressing Cyanoglobin were analyzed for gene expression and confirmed for Cyanoglobin protein expression. The E. coli cultures were grown overnight in LB medium with respective antibiotic concentrations and further used for induction of Cyanoglobin protein expression. The cultures were inoculated in about 10 mL of LB broth with about 5% inoculum concentration in two sets, one for gene expression analysis and other for protein expression analysis. The culture allowed to grow up to about 0.6 OD at 600 nm. The grown culture then induced with IPTG to a final concentration of about 1 mM. Induction was continued overnight at about 20° C. After about 18 hours cell pellet was collected by centrifugation at about 5000 rpm for about 5 minutes. Cell pellet was then used for RNA extraction and protein extraction.

### Gene Expression Analysis:

Cell pellet was resuspended in about 700 μl of RLT Buffer and mixed by vortexing the tube until the mixture became clear. RNA was extracted by RNeasy kit (Qiagen) as per manufacturer's instruction. Extracted RNA was then subjected to gel electrophoresis to check the quality of extracted RNA as shown in FIG. 14. Extracted RNA was then converted to cDNA using RevertAid cDNA synthesis kit following manufacturer's instruction. Gene specific primers were designed to check the expression of Cyanoglobin in recombinant clones. RPOS gene of E. coli was used as internal control for gene expression. Semi quantitative PCR using 10 ng of cDNA sample was then kept and expression was checked on agarose gel as shown in FIG. 15 which confirms overexpression of Cyanoglobin in E. coli.

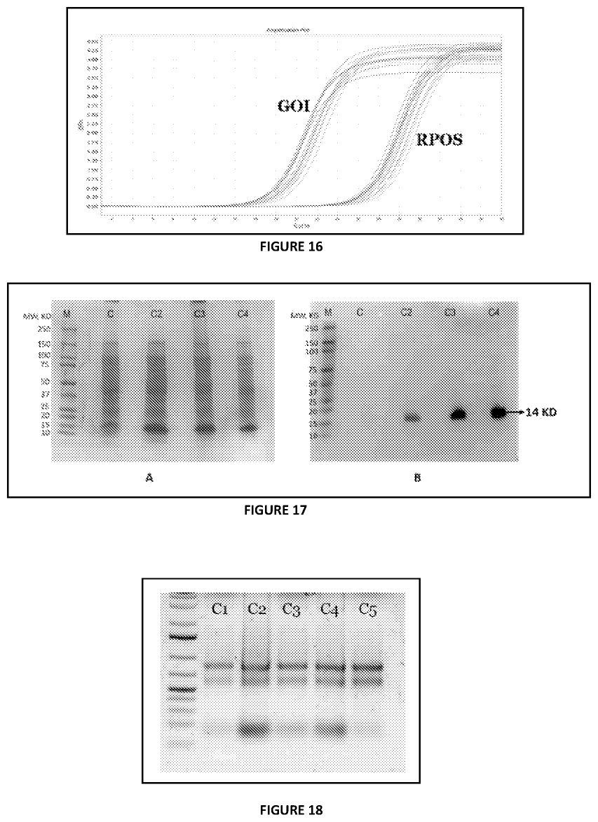

After confirming the overexpression of Cyanoglobin in E. coli by semiquantitative PCR, same optimized thermal condition was used for doing qRT PCR for gene expression analysis. Overexpression was clearly observed from amplification curves of gene of interest (GOI) and housekeeping (RPOS) gene (FIG. 16).

### Protein Expression Analysis:

Cell pellet was resuspended in PBS buffer (pH 7.2). The suspended pellet was subjected for sonication at about 50 kHz with pulse of about 10 sec. on and about 20 sec. off for about 2 minutes. Cell free lysate was prepared by centrifugation at about 5000 rpm for about 5 minutes and utilized for further analysis. Final concentration of about 15 μg of total proteins were separated on about 8% to about 16% polyacrylamide gels at about 120-150 V and transferred to PVDF membrane at about 11 V for about 1 hour. Then the PVDF membrane was blocked with about 5% solution of fat free skimmed milk. Cyanoglobin protein was probed with anti His antibody (primary antibody) followed by HRP conjugated secondary antibody. In between treatment of antibody the membrane was washed well with PBST (1×PBS and 0.04% Tween-20) and PBS (1×). Finally, the membrane was probed with ECL-HRP substrate for detection of Cyanoglobin protein band of molecular size about 14 KD (FIG. 17).

After confirmation of Cyanoglobin gene and protein expression in E. coli, one of the better performing clone was identified and used for further cloning. This clone (clone 3 in FIG. 17) was then cloned into Cyanobacterial cloning vector PMX 1848 and transformed into Cyanobacterium aponinum by homologous recombination and recombinant clones were screened for protein expression.

### Example 7: Expression Analysis of Cyanobacteria Strains Producing Cyanoglobin

Different strategies were used for cloning and expression of Cyanoglobin gene into Cyanobacterial strain Ls1. Recombinant clones for each of the strategy were screened for protein expression analysis. Cyanobacterial strains were analyzed for Cyanoglobin protein expression. Cryo stock of recombinant clone was streaked on BG11 agar plates and incubated for about 3-4 days in Percival under 12 h-12 h day light cycle. single colony was then inoculated into about 10 ml BG 11 media, which was then scaled up to 100 ml. About 20 ml culture was collected at OD 1, OD 2, OD 3, OD 4 and OD 6, and pelleted and further used to study optimized OD for gene expression. This optimization process was applied for all recombinant clones generated using different strategies.

### Gene Expression Analysis:

Frozen pellet of recombinant clone was used for expression analysis. The pellet was resuspended into nuclease free water and crushed to powder using liquid nitrogen. To the powdered pellet about 700 μl of Buffer RLT was added and RNA was extracted using RNeasy kit (Qiagen) following manufacturer's instruction. Extracted RNA was then subjected to gel electrophoresis to check the quality (FIG. 18). An intact band suggests good quality of extracted RNA. A smearing band typically suggests degraded RNA.

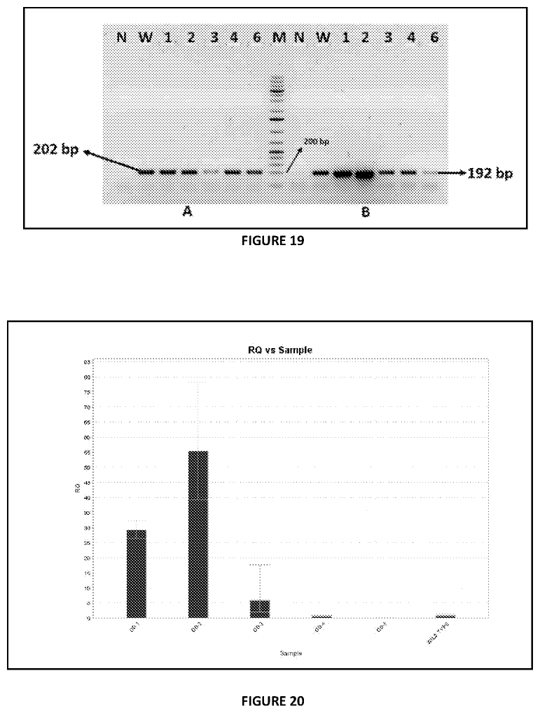

Extracted RNA was then converted to cDNA using RevertAid cDNA synthesis kit following manufacturer's instruction. Gene specific primers were designed to check the expression of Cyanoglobin in recombinant clones. RPOS gene of Cyanobacteria was used as internal control for gene expression. qRT PCR analysis was done to check the highest gene expression in Cyanobacteria. Semi quantitative PCR using about 10 ng of cDNA sample was then kept and expression was checked and confirmed on agarose gel as shown in FIG. 19.

qRT PCR analysis was also performed for samples collected at different stages (OD). Gene expression pattern is shown in FIG. 20. Better gene expression was observed at OD 2.

### Protein Expression Analysis:

Cell pellet was collected and resuspended in PBS buffer (pH 7.2). The suspended pellet was subjected for sonication at about 50 kHz with pulse of about 10 sec. on and 20 sec. off for about 8-10 min. Cell free lysate prepared by centrifugation at about 7500 rpm for about 10 minutes and utilized for further analysis. Final concentration of about 15 μg total proteins were separated on about 16% polyacrylamide gels at about 120-150V and transferred to PVDF membrane at about 11V for about 1 hour. Then the PVDF membrane was blocked with about 5% solution of fat free skimmed milk. Cyanoglobin protein was probed with anti His antibody (primary antibody) followed by HRP conjugated secondary antibody. In between the two antibody treatments the membrane was washed well with PBST (1×PBS and 0.04% Tween-20) and PBS (1×). After transfer the transferred protein was further fixed on the membrane by exposing it to glutaraldehyde. Finally, the membrane was probed with ECL-HRP substrate for detection of Cyanoglobin protein of molecular size of approximately 14 KD.

### Protein Expression Analysis of Clones Having Cyanoglobin Gene Under NirA Promoter—

In this strategy Cyanoglobin gene was cloned under NirA promoter into Sig-C neutral site of Ls01 strain and initially two clones were generated. The clones were screened for Cyanoglobin protein expression analysis by western blot. For protein expression analysis about 50 mg of total protein was loaded into about 16% PAGE gel.

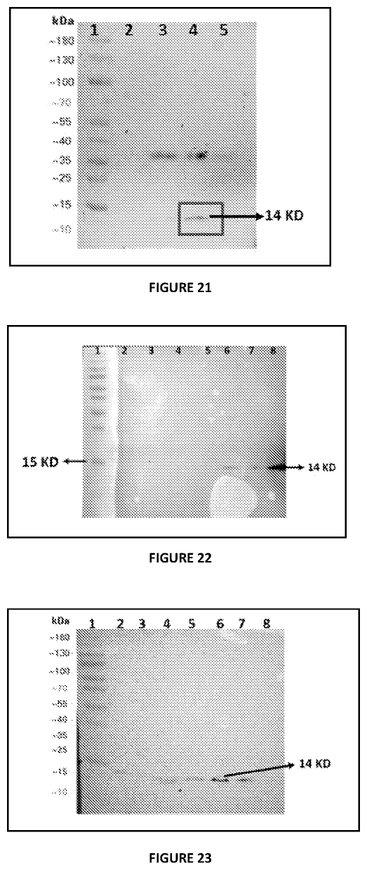

Protein expression was observed only in one clone under NirA promoter as band was observed at expected size of approximately 14 KD (marked in red box) (FIG. 21). Some more clones were generated later and analyzed for Cyanoglobin protein expression under NirA promoter shown in FIG. 22. After confirming the protein expression under NirA promoter, extra copy of Cyanoglobin gene was cloned under NirA promoter into the PsyB neutral site of the clone that showed protein expression under NirA promoter. Protein expression was then analyzed by western blot.

Higher Cyanoglobin protein expression was observed in the clone in which the Cyanoglobin gene was cloned under NirA promoter at PsyB site (this clone was having 3 copies of Cyanoglobin gene including native copy).

### Protein Expression Analysis of Clones Having Cyanoglobin Gene Under CpcB171 Promoter—

In this strategy Cyanoglobin gene was cloned under strong promoter CpcB171 at Sig-C site of strain Ls01 and two recombinant clones were generated. The clones were then analyzed for protein expression by western blot as described above.

Cpcb being a strong promoter, protein expression was observed in all the clones received (FIG. 23) with 2 clones having slightly higher protein band intensities than the rest. Based on the above screening, culture of one of the two clones was further scaled up and studied in simulated outdoor growth conditions (PBR).

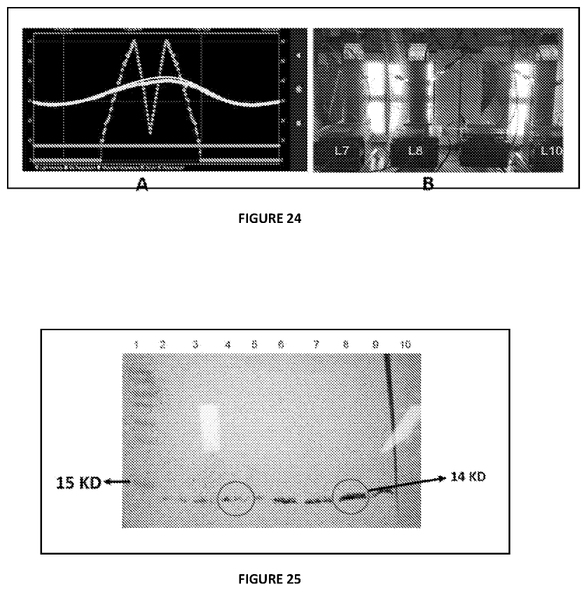

Said clone was grown in a photobioreactor (PBR) along with wild type Ls01 strain to check growth pattern and protein expression. The PBR simulated outdoor growth conditions with max light intensity reaching up to about 700 μmoles following M shaped curve prevalent in east west oriented PBRs. sinusoidal temperature model with maximum temperature up to about 42° C. at peak time interval and maintaining a constant pH of about 7.2 (FIG. 24). The cultures were inoculated in about 1 L of media with about 5% inoculum concentration and the PBR was operated in batch mode. Samples were collected at different growth stages OD-2, OD-4, OD-6 and OD-8, for gene and protein expression analysis.
c) Samples were collected at different growth stages OD 2, OD 4, OD 6 and OD 8 from the PBRs for protein expression. Protein bands were then detected by western blot FIG. 25.

At outdoor stimulated conditions higher expression was observed at OD 6 (marked with red circle) in the PBRs as shown in FIG. 25. These studies thus confirmed that Cyanoglobin protein expression can be obtained on a largescale even in outdoor growth conditions.

### Example 8: Determination of pI for Cyanoglobin Protein Using Offgel Apparatus Protocol

Lyophilized Cyanoglobin protein purified using affinity chromatography as described in the above examples was solubilized in milliQ water at the concentration of 3.15 mg/mL. 100 μg of the Cyanoglobin protein solution was loaded on a 24 well IPG strip (pH 3-10). A second sample of 100 μg of the Cyanoglobin protein solution was treated with 10 mM DTT for about 15 minutes at about 37° C. and loaded on a 24 well IPG strip (pH 3-10). The IPG strip rehydration buffer and the sample were prepared in about 1.25× stock solution using thiourea, DTT, Glycerol and offgel buffer. The default protocol over about 48 hours was used to run isoelectric focusing in the offgel apparatus. The samples were collected from each well after completion of the run and about 20 μL of each sample was loaded on the gel.

Formula used for determination of pH in each well—

$$= Starting pH of the IPG strip + \frac{pH difference in the IPG strip}{No . of wells - 1} \times ( Desired well no . - 1 )$$

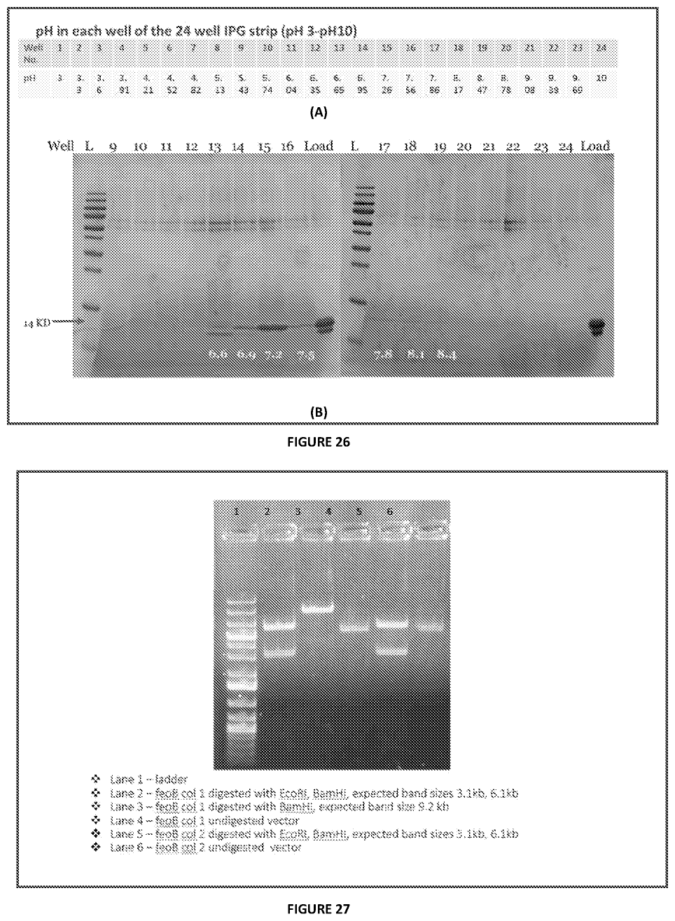

Results: The affinity purified Cyanoglobin sample was found to have both oxygenated and deoxygenated Cyanoglobin bands (as seen in the well showing load in FIG. 26B). The loaded sample was fractionated in a broad pH range from pH about 6.6 to 8.4. It was found that the major band of deoxygenated Cyanoglobin seen below the oxygenated Cyanoglobin band had a pI of about 6.6. However, the oxygenated Cyanoglobin band was fractionated in the well with pH 7.2 i.e. said band showed a pI of about 7.2. It was also observed that some minor bands got fractionated in adjacent wells due to very close pH in the wells and lower sensitivity of the instrument. The two bands observed at pH 6.6 and 7.2 on the gel therefore showed two forms—oxygenated and deoxygenated—of Cyanoglobin.

In addition to the above, it was also observed that an additional treatment with dithiothreitol (DTT) did not show any difference in the fractionation.

### Example 9: Construction of E. coli Strain Overexpressing Cyanoglobin, HemA and FeoB Using Recombinant Plasmid pPSRR24 (pET28a-Cyanoglobin-hemARs-FeoB)

FeoB gene codon optimized for E. coli (SEQ ID No. 20) was synthesized and used as a template for amplifying it such that the resulting amplicon had EcoRI, BamHI restriction sites at both the ends.

In parallel, E. coli NEBR 5-alpha cells harbouring the plasmid construct pET28a-Cyanoglobin-hemARs were cultivated and subjected to plasmid extraction using the plasmid miniprep kit (Qiagen, 27104). The obtained plasmid and the amplified FeoB gene were independently digested by using restriction enzyme EcoRI, BamHI and purified using the gel extraction kit (Qiagen, 28704). Both the linear DNA fragments were ligated using T4 DNA ligase (NEB, M0202 S) to result in plasmid pPSRR24 (SEQ ID No. 23) and transformed in competent E. coli NEB® 5-alpha cells (NEB, C2987I) to obtain transformants on LB agar plates containing about 50 μg/mL kanamycin.

The obtained transformants were screened for true positives by using diagnostic PCR. The confirmed recombinant strain (MTCC 25557) was preserved at about −80° C. as glycerol stock. The recombinant plasmid was isolated from the strain using the plasmid miniprep kit (Qiagen, 27104). FeoB positive clones were confirmed by restriction enzyme double digestion (FIG. 27). The recombinant plasmids were transformed in competent E. coli strain to obtain recombinant E. coli strain overexpressing Cyanoglobin, HemA and FeoB on LB agar plates containing about 50 μg/mL kanamycin.

For inducing protein expression, about 200 ml of Terrific broth (TB) medium was inoculated in a 1 litre flask with pre-inoculum of recombinant E. coli strain as obtained in Example 1 (overnight grown in TB medium from glycerol stock). The starting OD of this culture at λ_{600 nm} was about 0.05. The culture was grown at about 37° C. at about 200 rpm till the OD at λ_{600 nm} was about 2. Once the OD reached about 2, the culture was induced with about 1 mM IPTG and incubated at about 20° C. overnight.

After induction, the culture was pelleted down by centrifugation. The supernatant was decanted, and the cells were washed once with MQ water.

The cells were lysed using homogeniser/B-PER/sonication, wherein protease inhibitor and benzonase nuclease were added in the B-PER/buffer used for resuspending the pellet for homogenisation/sonication. The lysed cells were subjected to centrifugation obtain cell free lysate.

The cell free lysate was then subjected to affinity purification.

Expression analysis studies showed that FeoB co-expression along with HemA and cyanoglobin led to an improvement in the expression levels of cyanoglobin when compared to the recombinant E. coli strain of Example 1 co-expressing cyanoglobin and HemA.

### Example 10: Construction of Cyanobacterial Strain for HemA Overexpression Using Integrative Plasmid pPS23

HemARs gene codon optimized for the Cyanobacteria was synthesized (Invitrogen) and subsequently amplified along with overlapping ends compatible with prospective adjoining sequences. Similarly, regions upstream and downstream of the sigC locus, aadA selection marker and PnirA promoter were also amplified individually. All the amplified fragments were mixed as per the Gibson Assembly® cloning kit (NEB, E5510 S) protocol to result in E. coli transformants harboring the plasmid pPS23 (FIG. 4) on LB agar plates containing 100 μg/mL ampicillin. True positives were confirmed by using colony PCR followed by restriction digestion of the plasmid extracted using the plasmid miniprep kit (Qiagen, 27104). The recombinant E. coli strain construct PS57 (E. coli NEB® 5-alpha/pPS23) was preserved at −80° C. as glycerol stock.

The extracted plasmid pPS23 was further used for transformation of CphA and CphB KO Cyanobacterium aponinum strain as per the standardized protocol. The recombinants obtained on BG11 plates containing about 1.5 μg/mL each of spectinomycin and streptomycin were screened by colony PCR. The partially-segregated but confirmed recombinant Cyanobacterium aponinum strain was preserved at −80° C.

### Example 11: Preparation of a Food Grade Product Comprising the Cyanoglobin Produced in the Present Disclosure

A vegan meat substitute was prepared by combining a vegetable protein source, biomass derived from the present invention, a fat source, water, seasoning and optionally a binder and colouring agent. The components were mixed to form a dough and subjected to extrusion for texturization. A protein fibrous product emerged from the extruder wherein the extrusion imparted a structural form to the dough.

Without intending to be limited in scope by the following disclosure, the present disclosure provides examples of sequences that may be employed in the invention disclosed herein. Reference to the sequences by name in the description envisages employment of corresponding sequences as defined below or variants or fragments thereof.

1. A genetically modified bacterium expressing heme-containing protein comprising polynucleotide(s) encoding protein(s) selected from a group comprising HemA, HemL HemH and FeoB or any combination thereof.

2. The genetically modified bacterium as claimed in claim 1, wherein the bacterium is selected from E. coli and Cyanobacterium.

3. The genetically modified bacterium as claimed in claim 1, wherein the heme containing protein is an endogenous heme-containing protein or an exogenous heme-containing protein.

4. The genetically modified bacterium as claimed in claim 1, wherein the heme-containing protein is selected from a group comprising hemoglobin, cyanoglobin, and leghemoglobin.

5. The genetically modified bacterium as claimed in claim 1, wherein the heme-containing protein is endogenous or exogenous cyanoglobin.

6. The genetically modified bacterium as claimed in claim 1, wherein the genetically modified bacterium expresses endogenous cyanoglobin and comprises polynucleotide(s) encoding protein(s) selected from a group comprising HemA, HemL HemH and FeoB or any combination thereof.

7. The genetically modified bacterium as claimed in claim 6, wherein the genetically modified bacterium expresses endogenous cyanoglobin and comprises polynucleotide(s) encoding FeoB and protein(s) selected from a group comprising HemA, HemL, HemH; or
wherein the genetically modified bacterium expresses endogenous cyanoglobin and comprises polynucleotide(s) encoding FeoB and HemA.

8. (canceled)

9. The genetically modified bacterium as claimed in claim 6, wherein the bacterium is Cyanobacterium;
wherein the Cyanobacterium is engineered to knockout Cyanophycin synthetase (CphA) and Cyanophycinase (CphB) proteins.

10-11. (canceled)

12. The genetically modified bacterium as claimed in claim 9, wherein the genetically modified bacterium expresses exogenous cyanoglobin and comprises polynucleotide(s) encoding FeoB and protein(s) selected from a group comprising HemA, HemL and HemH.

13. The genetically modified bacterium as claimed in claim 9, wherein the genetically modified bacterium expresses exogenous cyanoglobin and comprises polynucleotide(s) encoding FeoB and HemA.

14. The genetically modified bacterium as claimed in claim 9, wherein the bacterium is E. coli or Cyanobacterium.

15. The genetically modified bacterium as claimed claim 1, wherein the polynucleotide(s) encoding protein(s) selected from a group comprising exogenous cyanoglobin, HemA, HemL, HemH and FeoB or any combination thereof are each under the control of the same or different promoter(s) selected from a group comprising Ppi1A, PT7, PnirA, Cpcb171 and PcpcB or any combination thereof.

16. A method of producing the genetically modified bacterium as claimed in claim 1, comprising—
a) Cloning genes encoding protein(s) selected from a group comprising HemA, HemL, HemH and FeoB or any combination thereof into recombinant DNA vector(s);
b) Transforming the recombinant DNA vector(s) into a bacterium expressing heme-containing protein,
to obtain the genetically modified bacterium.

17. The method as claimed in claim 14, wherein the bacterium expressing heme-containing protein expresses an endogenous heme-containing protein or is genetically modified to express an exogenous heme-containing protein.

18. The method as claimed in claim 14, wherein when the bacterium is genetically modified to express an exogenous heme-containing protein, the method comprises
a) Cloning genes encoding the exogenous heme-containing protein and protein(s) selected from a group comprising HemA, HemL, HemH and FeoB or any combination thereof into recombinant DNA vector(s);
b) Transforming the recombinant DNA vector(s) into the bacterium,
to obtain the genetically modified bacterium.

19. The method as claimed in claim 14, wherein the bacterium expressing heme-containing protein is engineered to knockout Cyanophycin synthetase (CphA) and Cyanophycinase (CphB).

20. (canceled)

21. The method as claimed in claim 18, wherein when the heme-containing protein is an endogenous heme-containing protein, the genetically modified bacterium is triggered to overexpress the endogenous heme-containing protein;
wherein the genetically modified bacteria is cultured in presence of aeration;
wherein the genetically modified bacteria is cultured in presence of a CO₂/air mixture of about 1% to about 4% (v/v) CO₂ in air; and/or
wherein the genetically modified bacteria is cultured at a temperature from about 15° C. to about 40° C.

22-24. (canceled)

25. The method as claimed in claim 18, wherein the genetically modified bacteria is cultured for a time-period ranging from about 5 hours to about 10 days;
wherein the genetically modified bacteria is cultured at a relative humidity of about 60% to about 80%;
wherein the genetically modified bacteria is cultured at a pH of about 6 to about 8; and/or
wherein the genetically modified bacteria is cultured at a pH of about 6 to about 8.

26-28. (canceled)

29. The method as claimed in claim 18, wherein the produced heme-containing protein is subjected to purification; and wherein the purification is performed by method(s) selected from a group comprising chromatography, heating, DCM method, PEG method and dual aqueous phase extraction or any combination thereof.

30. (canceled)

31. A pharmaceutical or food product comprising the genetically modified bacteria as claimed in claim 1 or the heme-containing protein produced therefrom.
